SEQUENCE LISTING (2 sequences)

SEQ ID NO: 1 - DNA, 20 bp, Artificial Sequence
  Primer
agagtttgatcctggctcag

SEQ ID NO: 2 - DNA, 19 bp, Artificial Sequence
  Primer
ggttaccttgttacgactt

(19) United States
(12) Patent Application Publication
(10) Pub. No.: US 2017/0106019 A1
(43) Pub. Date: Apr. 20, 2017

(54) COMPOSITIONS AND METHODS FOR MITIGATING DRUG RESISTANT BACTERIA

(71) Applicants: Shyam S. Mohapatra, Lutz, FL (US); Alya Limayem, Tampa, FL (US)

(72) Inventors: Shyam S. Mohapatra, Lutz, FL (US); Alya Limayem, Tampa, FL (US)

(73) Assignee: University of South Florida, Tampa, FL (US)

(21) Appl. No.: 15/298,832

(22) Filed: Oct. 20, 2016

Related U.S. Application Data
(60) Provisional application No. 62/243,877, filed on Oct. 20, 2015.

(51) Int. Cl. A61K 33/30; A61K 31/722; A61K 9/14

(52) U.S. Cl. CPC A61K 33/30; A61K 9/14; A61K 31/722

(57) ABSTRACT

The current invention is a broad-based remediation mechanism against MRFs and includes nanotechnology formulations and methodologies that may be used to develop novel mitigation strategies against certain drug resistant bacterial strains. In an embodiment, the current invention relates to mitigation of drug resistant bacteria from nosocomial infections, for example in hospitals and in food animals. The invention uses hybrid nanomaterials comprising oligo-chitosan and zinc oxide formulated as nanoparticles and micelles. The inventors unexpectedly found unique properties of very small oligomers of chitosan that effectively mitigate MRFs- and Vancomycin-Resistant Enterococcus (VRE)-induced illnesses without compromising the balance of the beneficial flora in the abdomen. Also, the combination of chitosan with zinc oxide demonstrated synergistic and unexpected effects in remediation of important food-borne bacteria including the resistant types.

### CROSS-REFERENCE TO RELATED APPLICATIONS

This nonprovisional application claims priority to U.S. Provisional Patent Application No. 62/243,877, entitled “Compositions and Methods of Mitigating Drug Resistant Bacteria”, filed Oct. 20, 2015, the entirety of which is incorporated herein by reference.

### BACKGROUND OF THE INVENTION

1. Field of the Invention

This invention relates, generally, to drug resistant bacteria. More specifically, it relates to mitigation of drug resistant bacteria from nosocomial infections, for example in hospitals and in food animals.

2. Brief Description of the Prior Art

Multidrug resistant gastrointestinal, fecal bacteria (MRF) are proliferating at a considerable rate to reach and impact downstream food chains, as well as hospital settings [1-4]. While there are several foodborne pathogens originating from animal gut having resistance, MRFs remain the leading cause of hospital-acquired infections.

Clonal complex resistant strains (CCs) of Enterococcus faecium, including mainly the multidrug resistant strain, have emerged as a leading cause of nosocomial pathogens, constituting a serious level of threat that has caused almost 20,000 infections and 1,300 deaths each year in the U.S. [5]. This MRF is now the primary cause of patient urinary tract and bloodstream infections in hospitals and could further lead to endocarditis [6] and death primarily in immunocompromised populations.

Conjointly in this pathogenic spread, Escherichia coli is implicated in millions of extra-intestinal infections, resulting in more than 100,000 cases of sepsis and 40,000 sepsis-associated deaths [7-9]. Moreover, the enormous intrinsic capability and phenotypic elasticity of the MRF strains—mainly E. faecium—enable them to acquire other genes from the environment, mutate continuously, and transfer genes to other pathogens, including primarily Salmonella and Campylobacter genera found in food animals [10-12]. Although some strains of E. faecium are used in the food industry and are also known for their probiotic attributes [13], the tremendous ability of some strains to acquire resistance would be a major bottleneck. It is also quite possible that resistant MRFs from food form a niche in a human's gastrointestinal tract, leading to a reservoir of resistance [14] and consequently jeopardizing the lives of the most immunocompromised populations [15].

While the mutating MRFs constitute a serious level of threat [5,16], some strains of E. faecium are also lactic acid bacteria and are known for their probiotic attributes. They have been extensively added in food for their fermentative ability and health benefits. It has been shown that rabbits in animal husbandries that were given water containing E. faecium as a probiotic had higher average weight gains as well as a healthier natural intestinal flora [17]. While E. faecium helps prevent antibiotic-associated diarrhea, enhance the immune system, and lower the cholesterol level [13], other strains are used for their food safety attributes in limiting zoonotic pathogens from food animals through bacteriocin production [13].

As evidenced by De Kwaadsteniet et al. (2005) [18], E. faecium P21 isolated from sausage produces both enterocins A and B. Enterocins proved to be active against a substantial range of Gram-positive bacteria including primarily Listeria species and Staphylococcus aureus. E. faecium, RZS C5 strain, has been isolated from natural cheese and also demonstrates anti-listerial properties without exhibiting virulence factors [19]. Nonvirulent strains of E. faecium have been suggested as a possible probiotic against microbes possessing antimicrobial resistances [13,20].

A substantial review on the antibacterial properties of bacteriocins has been implemented by Fisher and Phillips (2009) [21]. However, the tremendous ability of some strains to acquire virulence genes from other strains and convert into pathogenic strains would hinder the beneficial attributes of E. faecium. This is increasingly more problematic due to the considerable ability of E. faecium to mutate and acquire virulent genes in multiple types of environment [15].

Accordingly, what is needed is an effective intervention mechanism/therapy for mitigating or reducing multi-drug resistant pathogens found in food animals, humans, and the respective environment. However, in view of the art considered as a whole at the time the present invention was made, it was not obvious to those of ordinary skill in the field of this invention how the shortcomings of the prior art could be overcome.

While certain aspects of conventional technologies have been discussed to facilitate disclosure of the invention, Applicants in no way disclaim these technical aspects, and it is contemplated that the claimed invention may encompass one or more of the conventional technical aspects discussed herein.

The present invention may address one or more of the problems and deficiencies of the prior art discussed above. However, it is contemplated that the invention may prove useful in addressing other problems and deficiencies in a number of technical areas. Therefore, the claimed invention should not necessarily be construed as limited to addressing any of the particular problems or deficiencies discussed herein.

In this specification, where a document, act or item of knowledge is referred to or discussed, this reference or discussion is not an admission that the document, act or item of knowledge or any combination thereof was at the priority date, publicly available, known to the public, part of common general knowledge, or otherwise constitutes prior art under the applicable statutory provisions; or is known to be relevant to an attempt to solve any problem with which this specification is concerned.

### BRIEF SUMMARY OF THE INVENTION

The long-standing but heretofore unfulfilled need for compositions and methods for mitigating drug resistant bacteria is now met by a new, useful, and nonobvious invention.

In an embodiment, the current invention is a nanoparticle formulation for treating a nosocomial infection, comprising a combination of a therapeutically effective amount of chitosan and a therapeutically effective amount of zinc oxide.

In another embodiment, the current invention is a methodology for treating a nosocomial infection, comprising administering a combination of a therapeutically effective amount of chitosan and a therapeutically effective amount of zinc oxide.

These and other important objects, advantages, and features of the invention will become clear as this disclosure proceeds.

The invention accordingly comprises the features of construction, combination of elements, and arrangement of parts that will be exemplified in the disclosure set forth hereinafter and the scope of the invention will be indicated in the claims.

### BRIEF DESCRIPTION OF THE DRAWINGS

For a fuller understanding of the invention, reference should be made to the following detailed description, taken in connection with the accompanying drawings, in which:

FIG. 1 depicts an MIC test of chitosan alone.

FIG. 2 depicts an MIC test of ZnO alone.

FIG. 3 depicts an MIC test of CZNPs.

FIG. 4 depicts chitosan particles carrying zinc oxide on the collapsing outer membranes of resistant strains.

FIG. 5A depicts wild type bacteria in contact with zinc oxide particles, showing no adverse effects from exposure to the zinc oxide at 4 hours.

FIG. 5B depicts MRFs including E. coli BAA-2471 and E. faecium 1449 showing adsorbed chitosan on their cell membranes. The resistant bacteria have begun to lyse and collapse.

FIGS. 6A-6F depict effects of C₁ZNP_{S} on resistant E. coli BAA-2471 and E. faecium 1449 alone and co-cultured after 16 hours exposure.

FIG. 6A depicts E. coli BAA-2471 exposed to zinc oxide nanoparticles and chitosan for 16 hours. The bacterium on the left shows chitosan adsorbed to the cell membrane and zinc oxide nanoparticles attached to the membrane. Low electron density indicates that the bacterium is lysing. An intact E. coli BAA-2471 is on the right.

FIG. 6B depicts E. coli BAA-2471 exposed to chitosan and zinc oxide nanoparticles, showing adsorption of chitosan to cell membrane of bacteria and adherence of zinc oxide to bacteria. ZnO is causing pitting of the bacterial membrane.

FIG. 6C depicts E. faecium 1449 exposed to chitosan and zinc oxide. An intact E. faecium 1449 is in the lower left. Small high-density regions inside the cell are indicative of the nanoparticles. Presence of asterisk-like fragments detail lyses and disintegration of the cell wall.

FIG. 6D is a higher magnification photograph of the disintegration of the cell wall of E. faecium.

FIG. 6E depicts a resistant co-culture, MRF of E. faecium 1449 and E. coli BAA-2471 subjected to the combinatory nanoparticles. Nanoparticles surrounding the outside of the cells demonstrate clearly the formation of a synergistically formed meshing. Lower electron density indicates lyses of the bacteria, which is noticeable in both bacterial strains though notably more soon E. coli BAA-2471 in this panel.

FIG. 6F is a higher magnification photo of the co-cultured MRFs detailing the presence of nanoparticles surrounding and inside both bacterial species.

FIG. 7 depicts [16s rDNA]—Digested Waste (A-E) 11-10-2015 J.S.

FIGS. 8A-8F are TEM images after 24 h exposure of nanoparticles against microbial contaminants and algae.

FIG. 8A depicts Bacillus species exposed to zinc oxide nanoparticles and chitosan for 24 h. Low electron density indicate that a bacterium is lysing. High electron density within the lysing cells as indicated by the red arrow is representative of the nanoparticles inside the cell.

FIG. 8B depicts Bacillus culture exposed to chitosan and zinc oxide nanoparticles, showing adsorption of chitosan within the dying cell and adherence of the nanoparticle “meshing” to bacteria. A living cell of high electron density is visible to the right.

FIG. 8C depicts M. luteus subjected to the combinatory nanoparticles. The cell noted by the red arrow in the bottom of the cluster appears shriveled and its cell wall is forming protrusions. The nanoparticle meshing is visible at the top middle of the image.

FIG. 8D is a broader magnification photo of M. luteus detailing lysis of multiple cells. The low electron density of the cells indicate the cells are dying. Small high density particles are distinctive of the nanoparticles inside the lysed cell. High level of cellular protrusions indicates extensive damage to the Gram-positive cell wall.

FIG. 8E depicts a dying Pseudomonas cell. To the left, living M. luteus cells are present for reference. The red arrow points to a high electron density structure representing the nanoparticles within the bacterial cell. The low electron density of the cell indicates that the nanoparticles are degrading and damaging the cell membrane of the Gram-negative bacteria.

FIG. 8F depicts a single Pseudomonas cell above a series of dying, bottom left, and intact M. luteus cocci. The high electron density cocci species near the Pseudomonas cell are used as a reference. The cell indicated by the red arrow demonstrates the process of lysis when compared to the surrounding cells despite moderate electron density.

FIG. 8G depicts a single algae cell in front of a dying lysed Gram-negative bacteria. The high electron density of the spherical species when compared to the Gram negative bacteria indicate the cell is largely unaffected. Small scattered electron densities show the CZNPs outside of the meshing.

### DETAILED DESCRIPTION OF THE PREFERRED EMBODIMENT

In the following detailed description of the preferred embodiments, reference is made to the accompanying drawings, which form a part thereof, and within which are shown by way of illustration specific embodiments by which the invention may be practiced. It is to be understood that other embodiments may be utilized and structural changes may be made without departing from the scope of the invention.

As used in this specification and the appended claims, the singular forms “a”, “an”, and “the” include plural referents unless the content clearly dictates otherwise. As used in this specification and the appended claims, the term “or” is generally employed in its sense including “and/or” unless the context clearly dictates otherwise.

Continuous use and often overuse of traditional antibiotics has resulted in antibiotic resistance [22] and to the induction of bacteria that are unresponsive to a wide range of antibiotics (e.g., induction of the “Super Bug” that is unresponsive to a wide range of antibiotics [23]). Because some strains of multidrug resistant fecal bacterial—primarily Enterococcus—has exhibited the ability to develop resistance to most, if not all, drugs used against them [15], an effective antimicrobial that is natural/nontoxic, durable/sustainable, and cost-effective is needed and described herein.

The naturally occurring nanoparticle chitosan derived from crustacean shells has proven to be effective as an antibacterial against a variety of pathogens namely Gram-negative strains and biofilms in vitro [24]. Chitosan was also tested as a microbicide not only for its nontoxic and scalable attributes but also because of its potential to serve as a nanoparticle delivery system for other antimicrobials and plant extracts [25-27]. Therefore, it delivers the drug effects to the target cell with high potency and precision [28]. Aside from chitosan, zinc oxide (ZnO) is also emerging as an effective nanocomponent not only for its effectiveness against some cancer cells [29] but also for its potential to mitigate some Gram-positive strains with regard to its selective toxicity [29].

Specifically, the current invention is a broad-based remediation mechanism against MRFs and includes nanotechnology formulations and methodologies that may be used to develop novel mitigation strategies against these drug resistant strains. In an embodiment, the current invention relates to mitigation of drug resistant bacteria from nosocomial infections, for example in hospitals and in food animals. The invention uses hybrid nanomaterials comprising oligo-chitosan and zinc oxide formulated as nanoparticles and micelles.

While there are a few research studies that have demonstrated some antiseptic and chemical attributes of chitosan and ZnO jointly used in cotton fabric in addition to UV protection [30,31], little is known about their effectiveness against antibiotic resistant strains and MRFs-induced illnesses. The combination of chitosan oligomer and ZnO has an extraordinary antimicrobial potential compared to either agent alone and can be useful against a broad spectrum of multi-drug resistant pathogens in biomedical and food industry areas.

As will be shown herein, the inventors unexpectedly found unique properties of very small oligomers of chitosan that effectively mitigate MRFs- and Vancomycin-Resistant Enterococcus (VRE)-induced illnesses without compromising the balance of the beneficial flora in the abdomen. Also, the combination of chitosan with ZnO demonstrated synergistic and unexpected effects in remediation of important food-borne bacteria including the resistant types.

Experiment/Study 1 (Mitigation of Drug Resistant Bacteria)

The nosocomial MRF, Gram-negative-Escherichia coli, and Gram-positive-Enterococcus faecium are of prime concern to public health safety. This study was performed to examine effectiveness of a natural nanomicelle-based chitosan having different molecular weight and non-toxic ZnO combination to treat these MRF strains in vitro and in vivo. Herein, the antimicrobial effects of nanoparticles (NPs) of chitosan alone, ZnO alone, and chitosan and ZnO (CZNPs) in combination at 1:1 were examined on co-cultured MRFs through the minimal inhibitory concentration (MIC) test according to National Committee for Clinical Laboratory Standards (NCCLS). Toward improving the stability and activity of CZNPs, a carrier system was initially developed comprising PEG-functionalized phospholipid micelles. Using either gadolnium oxide in core and MnO with Dox in the core along with the DNA and chitosan on shell.

Results indicated that chitosan alone was ineffective at a concentration less than 5 mg/ml against either strains of bacteria as well as against the co-culture. Results further indicated that zinc oxide alone only showed effectiveness against E. faecium at a concentration of 3.125 mg/mL.

However, surprisingly, the MRF co-culture, E. coli BAA-2471 and E. faecium 1449 was completely inhibited by the CZNPs with an average minimal MIC of 0.781 mg/mL (low MW chitosan 1 of 3 KDa), and an average maximal MIC of 1.302 mg/mL (high MW chitosan 3 of 50 KDa). Synergism of (CZNPs) proved to be effective against MRF co-culture. This synergistic effect of chitosan and ZnO has proven to be far greater than what would be expected by a combination of the two

### A. MATERIALS AND METHODS

i. Sample Collection and Culture Conditions

The MRF, Gram-negative, E. coli ATCC; BAA-2471 purchased from ATCC and Gram-positive E. faecium 1449 provided by Moffitt Cancer Center were used as target bacteria for this study. These MRF strains were selected for their broad spectrum resistance and virulence. Only tigecycline was effective against these strains. The above isolates were cultured on Tryptic Soy Agar (TSA) and Tryptic Soy Broth (TSB) (Sigma, St. Louis, Mo.) and incubated at 37° C. for 24 hours and maintained at 37° C. for 24 hours. Bacterial growth was prepared by adding 100 μL fresh culture having 9.5×108 Colony Forming Units (CFU) on Tryptic Soy Agar (TSA) plates.

The chitosan nanoparticles with three different MWs were provided by Dr. Mohapatra's Lab, College of Pharmacy at the University of South Florida (gift of TRANSGENEX NANOBIOTECH Inc., Tampa). The ZnO was purchased from Sigma (SIGMA, St. Louis, Mo.).

ii. Serial Dilution and Plate Counts

Single colonies of E. coli BAA-2471 and E. faecium 1449 were inoculated from TSA plate to 15 mL TSB broth (Sigma, St. Louis, Mo.), respectively. Each broth culture was incubated at 37° C. for 24 hours. A seven times tenfold series dilution was performed by serially diluting 100 μL bacteria broth culture into 900 μL TSB. The 100 μL of each dilution was plated on TSA and incubated at 37° C. for 24 hours.

iii. Minimal Inhibitory Concentration (MIC) Assays

These overnight bacteria cultures were diluted 1 in 1,000 in fresh TSB or LB. Sterile 96-well plates were loaded with a co-cultured E. coli BAA-2471 and E. faecium 1449 (5×10⁵ cfu/mL) and screened against chitosan, ZnO alone and CZNPs at decreasing concentrations by 1:2 dilution to equal a total volume of 100 μL in each of the wells. All assays were performed in triplicate with identical results. Care was taken to not add more than 1.0% (CZNPs) to any well. The plates were then incubated at 37° C. overnight. The MICs were determined after 24 hours by visual determination of the minimum concentration of compound to inhibit growth. Inhibition of growth was determined by lack of turbidity in the wells.

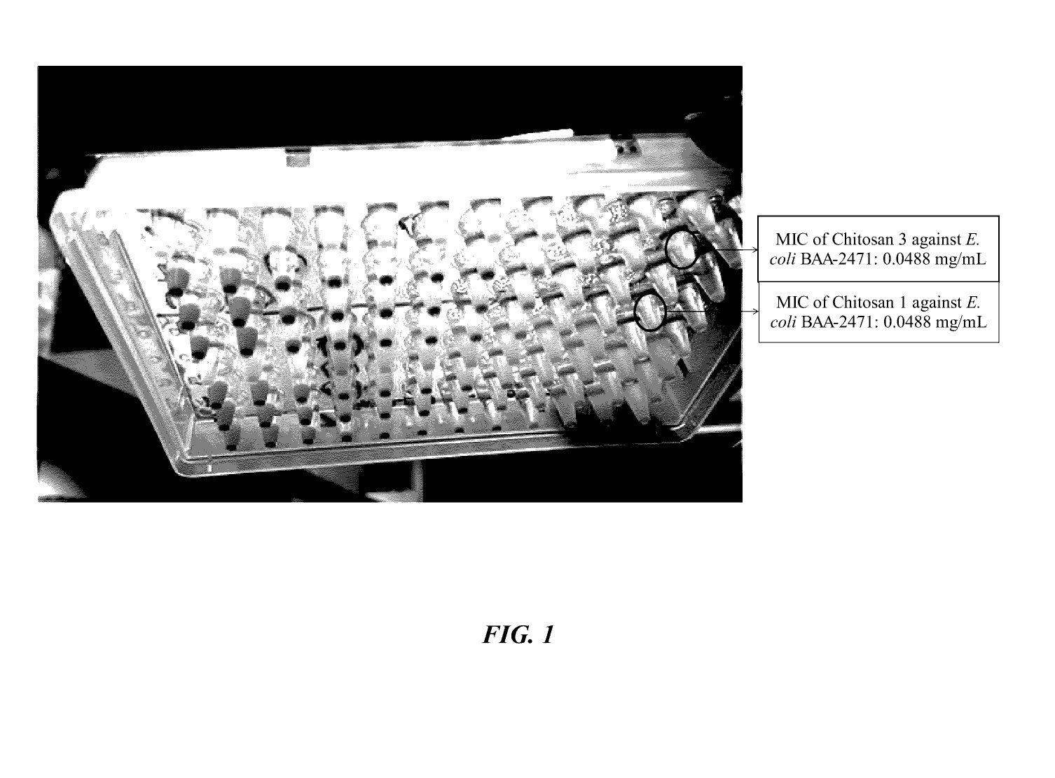

### B. CHITOSAN ALONE (FIG. 1)

The antimicrobial efficacies of chitosan tested after 24 hours incubation at 37° C. are shown in Table 1. Chitosan 1 and 3 was not effective against both E. faecium culture and the co-culture as well as their WT cultures. However, chitosan 1 and 3 showed a significantly inhibitory antimicrobial ability (0.0488 mg/mL) against multidrug resistant E. coli BAA-2471 culture compare to non-effective against WT E. coli MCC 13 culture.

TABLE 1
Minimum Inhibitory Concentration (MIC) of Chitosan against multidrug resistant fecal
flora and their wild type (WT) counterparts.

| | Strains of multidrug resistant with their WT counterparts | | | | | |
|---|---|---|---|---|---|---|
| | | | | Enterococcus | Co-culture | Co-culture |
| | Escherichia | Escherichia | | faecium | of BAA- | of MCC 13 |
| Antimicrobial | coli BAA- | coli MCC | Enterococcus | ATCC | 2471 and | and ATCC |
| NPs | 2471 | 13 (WT) | faecium 1449 | 35667 (WT) | 1449 | 35667 (WT) |

MIC presented in mg/mL

| | | | | | | |
|---|---|---|---|---|---|---|
| Chitosan 1 | <0.0488 | ND (>50) | ND (>50) | ND (>50) | ND (>50) | ND (>50) |
| (3 KDa) | | | | | | |
| Chitosan 3 | 0.0488 | ND (>50) | ND (>50) | ND (>50) | ND (>50) | ND (>50) |
| (50 KDa) | | | | | | |

*ND = Not Detected

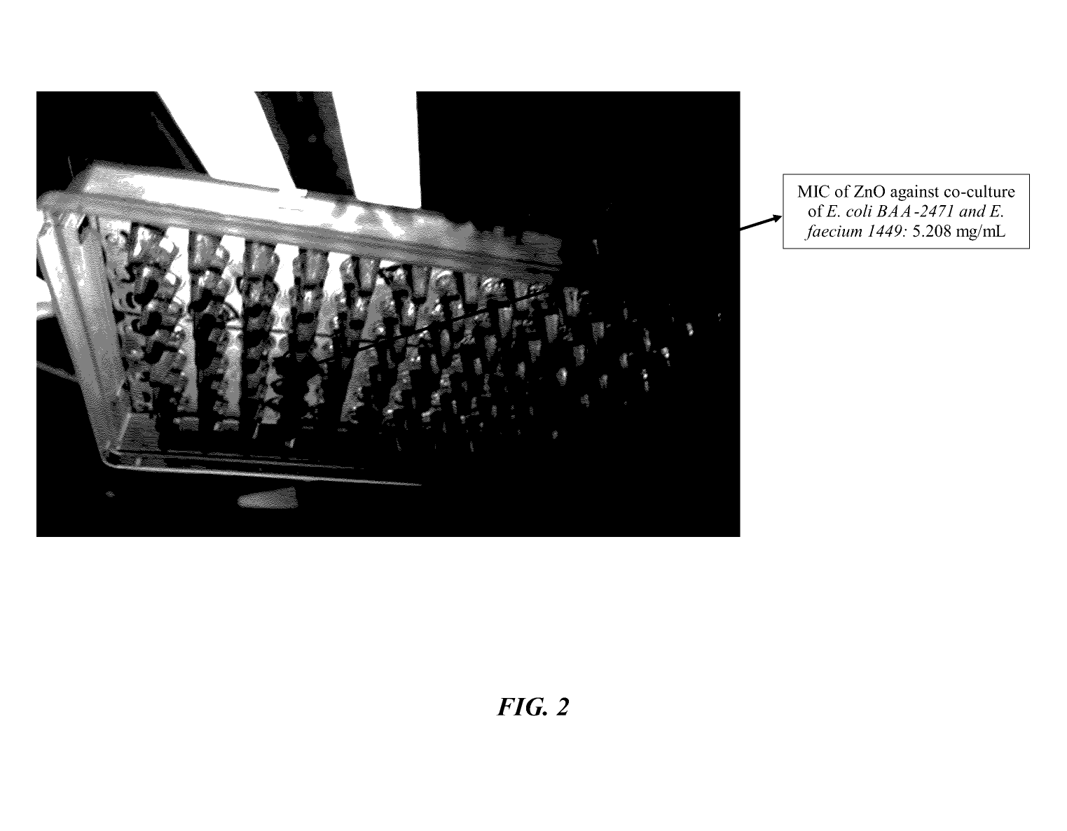

### C. ZNO ALONE (FIG. 2)

The MIC values of ZnO tested after 24 hours incubation at 37° C. are shown in Table 2. ZnO exhibited varying MICs against different cultures. According to the results, ZnO was more effective against WT E. coli MCC 13 (7.292 mg/mL) and WT E. faecium ATCC 35667 (0.391 mg/mL) than resistant E. coli BAA-2471 (13.54167 mg/mL) and E. faecium 1449 (3.125 mg/mL), respectively. However, ZnO showed a slightly lower MIC of 5.208 mg/mL against resistant co-culture than the WT co-culture with a MIC of 6.25 mg/mL.

TABLE 2
Minimum Inhibitory Concentration (MIC) of ZnO against multidrug resistant fecal flora
and their wild type (WT) counterparts.

| | Strains of multidrug resistant with their WT counterparts | | | | | |
|---|---|---|---|---|---|---|
| | | | | Enterococcus | Co-culture | Co-culture |
| | Escherichia | Escherichia | | faecium | of BAA- | of MCC 13 |
| Antimicrobial | coli BAA- | coli MCC | Enterococcus | ATCC | 2471 and | and ATCC |
| NPs | 2471 | 13 (WT) | faecium 1449 | 35667 (WT) | 1449 | 35667 (WT) |

MIC presented in mg/mL

| | | | | | | |
|---|---|---|---|---|---|---|
| ZnO | 13.54167 | 7.292 | 3.125 | 0.391 | 5.208 | 6.25 |

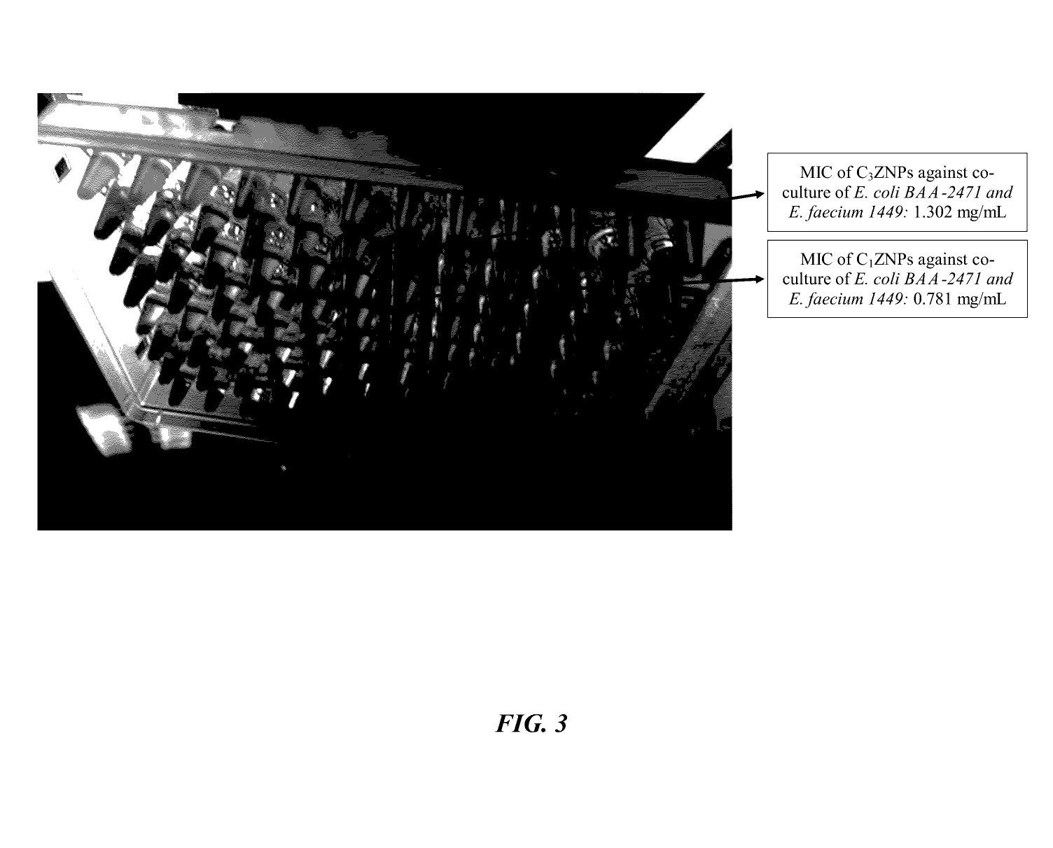

### D. CZNP (FIG. 3)

The antimicrobial efficacies of synergism of the combination of chitosan and ZnO tested after 24 hours incubation at 37° C. are shown in Table 3. Both C₁ZNPs and C₃ZNPs presented markedly higher antimicrobial efficacy against resistant cultures than WT cultures. The MIC values of C₁ZNPs against resistant E. coli BAA-2471, E. faecium 1449 and co-culture were 0.0488, 1.563 and 0.781 mg/mL, respectively. The MIC values of C₃ZNPs against resistant E. coli BAA-2471, E. faecium 1449 and co-culture were 0.0488, 1.042 and 1.302 mg/mL, respectively. Interestingly, C₁ZNPs with low MW (3 KDa) were much more effective than C₃ZNPs with high MW (50 KDa) against resistant co-culture. The MW was one of key factors of NPs to impact the antimicrobial efficacy against bacteria cultures.

TABLE 3
Minimum Inhibitory Concentration (MIC) of synergism of chitosan and ZnO against
multidrug resistant fecal flora and their wild type (WT) counterparts.

| | Strains of multidrug resistant with their WT counterparts | | | | | |
|---|---|---|---|---|---|---|
| | | | | Enterococcus | Co-culture | Co-culture |
| | Escherichia | Escherichia | | faecium | of BAA- | of MCC 13 |
| Antimicrobial | coli BAA- | coli MCC | Enterococcus | ATCC | 2471 and | and ATCC |
| NPs | 2471 | 13 (WT) | faecium 1449 | 35667 (WT) | 1449 | 35667 (WT) |

MIC presented in mg/mL

| | | | | | | |
|---|---|---|---|---|---|---|
| C₁ZNPs | <0.0488 | 2.604 | 1.563 | 3.125 | 0.781 | 2.083 |
| C₃ZNPs | <0.0488 | 2.083 | 1.042 | 2.604 | 1.302 | 3.125 |

* C₁ZNPs = chitosan 1 + ZnO
C₃ZNPs = chitosan 3 + ZnO

### E. REFERENCES

1. Bakhshi S, Padmanjali K S, Arya L S, 2008. Infections in childhood acute lymphoblastic leukemia: an analysis of 222 febrile neutropenic episodes. Pediatr Hematol Oncol 25: 385-392.
2. Cattaneo C, Quaresmini G, Casari S, Capucci M A, Micheletti M, et al., 2008. Recent changes in bacterial epidemiology and the emergence of fluoroquinolone-resistant Escherichia coli among patients with haematological malignancies: results of a prospective study on 823 patients at a single institution. J Antimicrob Chemother 61: 721-728.
3. Krcmery V, Spanik S, Mrazova M, Trupl J, Grausova S, et al., 2002. Bacteremias caused by Escherichia coli in cancer patients-analysis of 65 episodes. Int J Infect Dis 6: 69-73.
4. Limayem A, Donofrio R S, Zhang C, Haller E, Johnson M G, 2015. Studies on the drug resistance profile of Enterococcus faecium distributed from poultry retailers to hospitals, Journal of environmental science and health Part B Pesticides food contaminants and agricultural wastes. Accepted.
5. Centers for Disease Control and Prevention (CDC), 2013. Antibiotic Resistance Threats in the United States, 2013.
6. Lebreton F, van Schaik W, Manson McGuire A, Godfrey P, Griggs A, Mazumdar V, Corander J, Cheng L, Saif S, Young S, Zeng Q, Wortman J, Birren B, Willems R J L, Earl A M, Gilmore M S, 2013. Emergence of Epidemic Multidrug-Resistant Enterococcus faecium from Animal and Commensal Strains. mBio vol. 4 no. 4 e00534-13. doi: 10.1128/mBio.00534-13.
7. Eisenstein B I, Jones G W, 1988. The spectrum of infections and pathogenic mechanisms of Escherichia coli. Adv Intern Med 33: 231-252.
8. Johnson J R, Russo T A, 2002. Extraintestinal pathogenic Escherichia coli: “the other bad E. coli”. J Lab Clin Med 139: 155-162.
9. Orskov I, Orskov F, 1985. Escherichia coli in extra-intestinal infections. J Hyg (Lond) 95: 551-575.
10. Johnson J R, 1991. Virulence factors in Escherichia coli urinary tract infection. Clin Microbiol Rev 4: 80-128.
11. Johnson J R, Kuskowski M A, Gajewski A, Sahm D F, Karlowsky J A, 2004. Virulence characteristics and phylogenetic background of multidrug-resistant and antimicrobial-susceptible clinical isolates of Escherichia coli from across the United States, 2000-2001. J Infect Dis 190: 1739-1744.
12. Johnson J R, Stell A L, 2000. Extended virulence genotypes of Escherichia coli strains from patients with urosepsis in relation to phylogeny and host compromise. J Infect Dis 181: 261-272.
13. Franz C M, Huch M, Abriouel H, Holzapfel W, Galvez A, 2011. Enterococci as probiotics and their implications in food safety. Int J Food Microbiol 151: 125-140.
14. Werner G, Klare I, Heier H, Hinz K H, Bohme G, Wendt M, and Witte W, 2000. Quinupristin/dalfopristin-resistant enterococci of the satA (vatD) and satG (vatE) genotypes from different ecological origins in Germany. Microb Drug Resist 6: 37-47.
15. Arias C A and Murray B E, 2012. The rise of the Enterococcus: beyond vancomycin resistance. Nat Rev Microbiol 10: 266-278.
16. Limayem A, 2015. The mutating gastrointestinal flora, multidrug resistant Enterococcus faecium, Agriculture, Food and Analytical Bacteriology. Accepted.
17. Laukova, A., L. Chrastinova, I. Placha, V. Strompfova, R. Szabóova, K. Cobanova, M. Chrenkova, Z. Formelova, J. Imrichova, J. Ha dryova, L. Ondruska, R. Jurck, R.
Ziitnan. 2012. Beneficial effect of bacteriocin-producing strain Enterococcus faecium EF 55 of non rabbit origin in rabbits. In: Hacklander, K. and Thurner, C. Ed. BOKU-Reports on wildlife research and game management. Proc 4th World Lagomorph Conference, Vienna, Austria, Vienna, p. 77.
18. De Kwaadsteniet, M., S. D. Todorov, H. Knoetze, L. M. Dicks. 2005. Characterization of a 3944 Da bacteriocin, produced by Enterococcus mundtii ST15, with activity against Gram-positive and Gram-negative bacteria. Int. J. Food Microbiol. 105:433-444.
19. Leroy, F., M. R. Foulquie Moreno, L. De Vuyst. 2003. Enterococcus faecium RZS C5, an interesting bacteriocin producer to be used as a co-culture in food fermentation. Int. J. Food Microbiol. 88:235-240.
20. Lund, B., C. Edlund. 2001. Probiotic Enterococcus faecium strain is a possible recipient of the vanA gene cluster. Clin. Infect. Dis. 32:1384-1385.
21. Fisher, K., C. Phillips. 2009. The ecology, epidemiology and virulence of Enterococcus. Microbiol. 155:1749-1757.
22. Sahoo S K, Parveen S, Panda J J, 2007. The present and future of nanotechnology in human health care. Nanomedicine 3:20-31.
23. Arias C A, Murray B E, 2009. Antibiotic-resistant bugs in the 21st century—a clinical super-challenge. N Engl J Med 360:439-443.
24. Paul S, Seema N P, 2015. In vitro antibacterial potential of chitosan and its derivatives on pathogenic enterobacteriaceae. Natl J Physiol Pharm Pharmacol 5: 119-124.
25. Reddy K M, Feris K, Bell J, Wingett D G, Hanley C, Punnoose A, 2007. Selective toxicity of zinc oxide nanoparticles to prokaryotic and eukaryotic systems. Appl Phys Lett 90:2139021-2139023.
26. Jin T, Sun D, Su J Y, Zhang H, Sue H J, 2009. Antimicrobial efficacy of zinc oxide quantum dots against Listeria monocytogenes, Salmonella Enteritidis, and Escherichia coli O157:H7. J Food Sci 74:M46-52.
27. Dixit S et al., 2013. Phospholipid micelle encapsulated gadolinium oxide nanoparticles for imaging and gene delivery. RSC Adv 3: 2727-2735.
28. Kumar A, Glaum M, El-Badri N, Mohapatra S, Haller E, Park S, Patrick L, Nattkemper L, Vo D, Cameron D F, 2011. Initial observations of cell-mediated drug delivery to the deep lung. Cell Transplant 20: 609-618.
29. Premanathan M, Karthikeyan K, Jeyasubramanian K, Manivannan G, 2011. Selective toxicity of ZnO nanoparticles toward Gram-positive bacteria and cancer cells by apoptosis through lipid peroxidation. Nanomedicine. 7:184-92.
30. Abd Elhady, M M. 2012. Preparation and characterization of chitosan/zinc oxide nanoparticles for imparting antimicrobial and UV protection to cotton fabric. International journal of carbohydrate chemistry, 2012: 6.
31. Perelshtein I, Ruderman E, Perkas N, Tzanov T, Beddow J, Joyce E, Gedanken, A, 2013. Chitosan and chitosan-ZnO-based complex nanoparticles: formation, characterization, and antibacterial activity. J Mater Chem B 1: 1968-1976.

All referenced publications are incorporated herein by reference in their entirety. Furthermore, where a definition or use of a term in a reference, which is incorporated by reference herein, is inconsistent or contrary to the definition of that term provided herein, the definition of that term provided herein applies and the definition of that term in the reference does not apply.

Experiment/Study 2 (Nanotherapeutics for Mutating Multi-Drug Resistant Fecal Bacteria)

Multi-drug resistant fecal bacteria (MRF) including gram-negative Escherichia coli and -positive Enterococcus faecium are of prime concern to food safety and public health [1-4]. This study was performed to test efficacy of a natural antimicrobial, polymeric chitosan-based nanoparticles combined with ZnO to in situ intervention. Herein, the effects of nanoparticles (NPs) of chitosan, ZnO alone, and a combination of chitosan and ZnO (CZNPs) at 1:1 were examined on co-cultured nosocomial MRFs and a wild type (WT) through the minimal inhibitory concentration (MIC) test conform to National Standards, NCCLS. Toward elucidating visually the mechanistic effects of NPs alone and CZNPs on MRF and WT strains, Transmission Electronic Microscopy (TEM) was performed. While chitosan 1 (C₁) and 2 (C₂) alone with a molecular weight of 3 kDa and 50 kDa, respectively inhibited resistant E. coli strain (E. coli BAA-2471), they were ineffective at a concentration less than 5 mg/mL on either E. faecium strains and the co-cultures. ZnO and chitosan alone did not exhibit optimal effects on MRF strains and cultures alone. However, the MRF co-culture, E. coli BAA-2471 and E. faecium 1449 was completely inhibited by the C₁ZNPs with an average minimal MIC of 0.781 mg/mL, and a maximal MIC of 1.302 mg/mL. Synergism of C₁ZNPs over C₂ZNPs proved to be predominantly inhibitive of MRF over WT co-cultures. Further TEM analyses demonstrated attachment and lysis of MRFs at 16 h past treatment. Conclusively, CZNPs inhibit MRF co-cultures and is a promising in vivo intervention agent.

### A. INTRODUCTION

Currently, some strains of multidrug resistant E. faecium concurrently with E. coli pose a serious level of threat to humans [1-4]. While there are several resistant foodborne pathogens originating from animal gut, some strains of MRFs including Vancomycin Resistant E. faecium (VRE) remain the leading cause of hospital-acquired infections with 10,000 hospitalized cases and 650 deaths each year in the U.S. [5]. Clonal complex 17 (CC17) is now the prime causative of patient urinary tract infections in clinical settings and could further lead to serious complications primarily in patients with long stay in hospitals [6]. Conjointly in this pathogenic spread, E. coli is implicated in millions of extraintestinal infections resulting in more than 100,000 cases of sepsis and 40,000 sepsis-associated deaths [7-9]. Moreover, phenotypic elasticity of these MRF strains mainly E. faecium enable them exchange genes with other pathogens such as Salmonella and Campylobacter genera found in food animals [10-12]. It is also quite possible that resistant MRFs from food animals form a niche in the humans' gastrointestinal tract leading to a reservoir of resistance [14], consequently, jeopardizing the lives of the most immunocompromised populations [15].

Despite the increasing threat of mutating MRFs [5,16], many strains of E. faecium are useful lactic acid bacteria that have been extensively added in food applications for their fermentative ability and health benefits. Despite their probiotic attributes [13], the considerable ability of some E. faecium strains to mutate in multiple types of environments has made the use of E. faecium as a fermentative strain questionable [15]. Furthermore, the continuous use of the traditional antibiotics has led to the induction of “Super Bugs” that are unresponsive to a wide range of antibiotics [22,23]. Enterococcus exhibited the ability to develop resistance to basically every drug used against them [15]. Thus, developing remediation strategies against these multi-drug resistant bacteria remains a major unmet need.

As such, an alternative natural antimicrobial that is nontoxic and sustainable was deemed of prime interest. Chitosan, arguably the most important derivative of chitin, is emerging as a strong, natural antimicrobial that is considered safe for human health [6]. Additionally, chitosan with its exposed —NH2 groups is involved in specific interactions with metals [6] which not only allows chitosan to behave differently from other polysaccharides due to the positive charge on its surface. In a solid state, chitosan is a semicrystalline polymer that is soluble in acidic solutions, however, its solubility depends on the distribution of acetyl groups along its polysaccharide chain and the molecular weight [7]. Chitosans have proven to be effective as an antibacterial against a variety of pathogens namely gram-negative strains and biofilms [24]. The effectiveness of such antibacterial activity was found in a previous report to be correlated with chitosan's molecular weight, thus allowing the chitosan biopolymers to be highly effective when compared to chitosan oligomers, which were significantly smaller [32].

Aside from Chitosan, Zinc oxide (ZnO) is emerging as an effective nanocomponent for its effectiveness against some cancer cells [29] in addition to its potential to mitigate some gram-positive strains with regard to its selective toxicity [29]. On the other hand, Zinc oxide nanoparticles seem to inhibit or cause bacterial death more efficiently when they are smaller in size but higher in concentration. A smaller size at higher concentrations provides higher specific surface areas and facilitates the penetration of the antimicrobial agent into the bacterial membrane [5].

A few studies demonstrated antiseptic and chemical attributes of chitosan and ZnO jointly used in cotton fabric in addition to UV protection [30,31], suggesting that a combination of chitosan oligomer and ZnO may provide better antiseptic property compared to either agent alone. However, little is known about the effectiveness of either of these nanoparticles against antibiotic resistant strains and MRFs induced illnesses.

We have been investigating the means of remediation of multi-drug resistant bacteria. Based on the reports that a combination of chitosan and ZnO may lead to better nanoformulations exhibiting antibacterial properties, in this study, the antibacterial properties of polymeric chitosan-based nanoparticles and/or ZnO was investigated and the results show that very small oligomers of chitosan effectively mitigate MRFs and VRE. The results for the first time demonstrates that chitosan oligomeric nanoparticles by themselves or in combination with ZnO provide for the effective remediation of MRFs and VRE.

### B. MATERIALS AND METHODS

i. Sample Collection and Culture Conditions

The MRF, gram-negative, E. coli BAA-2471 purchased from ATCC and gram-positive E. faecium 1449 provided from Moffitt Cancer Center were used as target bacteria for this study. These MRF strains were selected for their broad-spectrum resistance and virulence. Tigecycline was the only available drug effective against these strains. The above isolates were cultured on Tryptic Soy Agar (TSA) and Tryptic Soy Broth (TSB) (Sigma, St. Louis, Mo.) and incubated at 37° C. for 24 hours and maintained at 37° C. for 24 hours. Bacterial growth was prepared by adding 100 uL fresh culture having 9.5×108 Colony Forming Units (CFU) on Tryptic Soy Agar (TSA) plates.

The chitosan nanoparticles with three different MWs were provided by Dr. Mohapatra's lab in College of Pharmacy at USF. The ZnO was purchased from Sigma (Sigma, St. Louis, Mo.).

ii. Serial Dilution and Plate Counts

Single colonies of E. coli BAA-2471 and E. faecium 1449 were inoculated from TSA plate to 15 mL TSB broth (Sigma, St. Louis, Mo.), respectively. Each broth culture was incubated at 37° C. for 24 hours. A seven times tenfold series dilution was performed by serially diluting 100 μL bacteria broth culture into 900 μL TSB. The 100 uL of each dilution was plated on TSA and incubated at 37° C. for 24 hours.

iii. Minimal Inhibitory Concentration (MIC) Assays

These overnight bacteria cultures were diluted 1 in 1,000 in fresh TSB or LB. Sterile 96-well plates were loaded with a co-cultured E. coli BAA-2471 and E. faecium 1449 (5×10⁵ cfu/mL) and screened against chitosan, ZnO alone and CZNPs at decreasing concentrations by 1:2 dilution to equal a total volume of 100 μL in each of the wells. All assays were performed in triplicate with identical results. Care was taken to not add more than 1.0% (CZNPs) to any well. The plates were incubated at 37° C. overnight. The MICs were determined after 24 hours by visual determination of the minimum concentration of compound to inhibit growth. Inhibition of growth was determined by lack of turbidity in the wells.

iv. TEM Assay

Routine preparation of bacteria by negative staining would require pelleting the bacteria to rinse them in order to remove growth media protein through rinsing. Exposing unfixed bacteria to high-speed centrifugation could alter damaged surface membrane structure. Aldehydes, typically employed to fix and stabilize bacteria prior to observation in the electron microscope, could not be used to stabilize bacteria, as the aldehydes would crosslink proteins in the growth media to the bacterial surface, obscuring surface damage to the bacterial membranes, if present. A new method of fixing the bacteria in osmium tetroxide prior to pelleting was employed to stabilize the bacterial membranes. Osmium would not crosslink any protein in the culture medium to the bacteria, but would preserve the membrane structure of the bacteria throughout the centrifugation process, allowing rinsing to remove the culture media proteins necessary to prepare the bacteria for TEM, and impart electron density similar to that of uranyl acetate or other negative stains used to observe bacteria in the electron microscope.

Aliquots of bacteria in growth media were initially fixed in equal volume of 2% osmium tetroxide in distilled water for 10 minutes at 4° C. Following fixation, the bacteria were rinsed in distilled water and pelleted at 5000 RPM for 10 minutes. This rinse step was repeated three times. A proper dilution of bacteria was obtained to yield approximately 2000-3000 bacteria per drop, and one drop of a sample was applied to a carbon-formvar coated copper grid. The grid was allowed to air dry. This procedure was repeated for each sample. Once dry, the grids were observed and photographed in the electron microscope.

### C. RESULTS

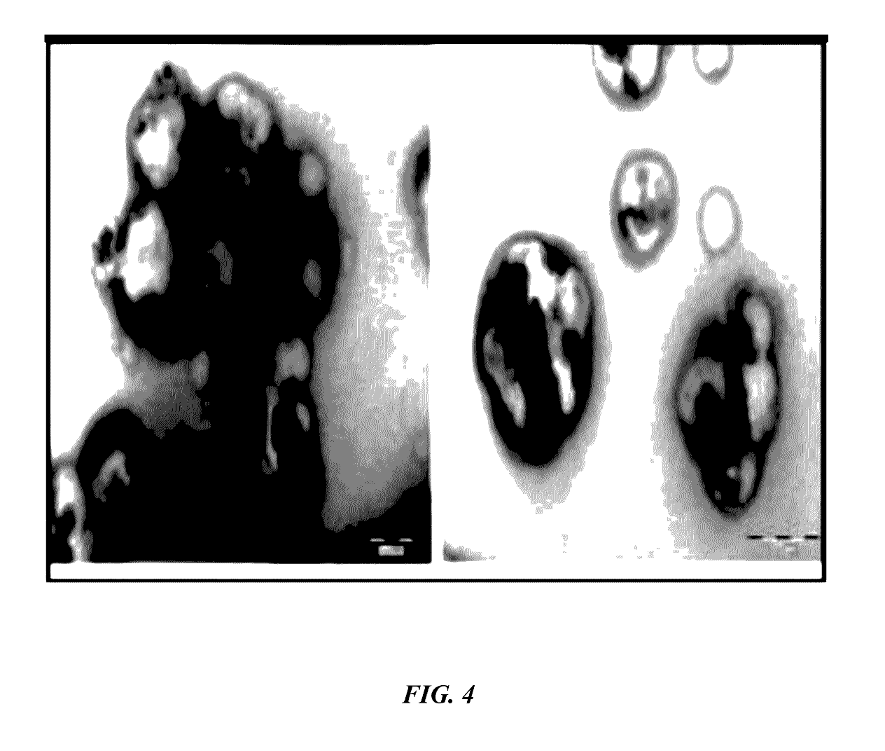

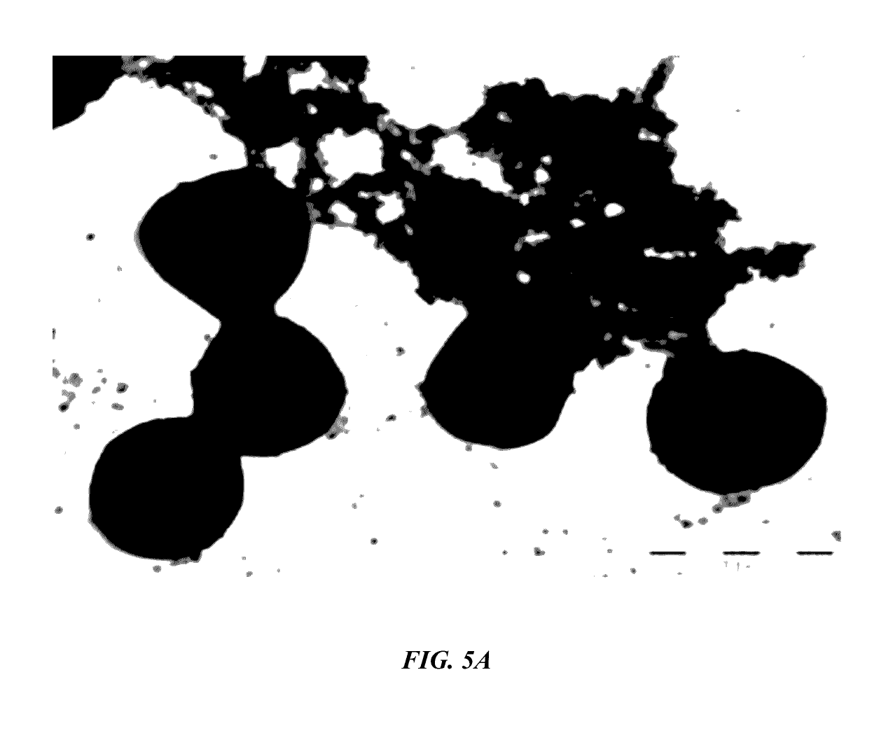

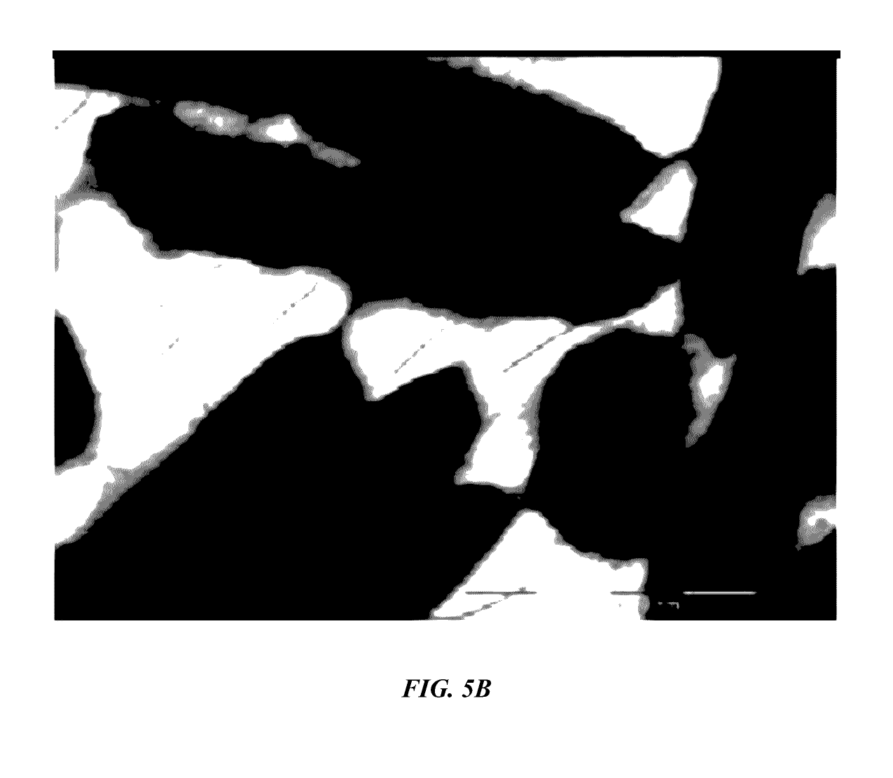

The antimicrobial efficacies of chitosan tested after 24 hours incubation at 37° C. are shown in Table 4. The chitosan 1 and 2 was not effective against both E. faecium culture and the co-culture as well as the WT cultures. However, chitosan 1 and 2 showed a significantly inhibitory antimicrobial ability (0.0488 mg/mL) against multidrug resistant E. coli BAA-2471 culture compare to non-effective against WT E. coli MCC 13 culture (FIG. 4). The MIC values of ZnO tested after 24 hours incubation at 37° C. are shown in Table 4. ZnO exhibited varying MICs against different cultures. According to the results, ZnO was more effective against WT E. coli MCC 13 (7.292 mg/mL) and WT E. faecium ATCC 35667 (0.391 mg/mL) than resistant E. coli BAA-2471 (13.54167 mg/mL) and E. faecium 1449 (3.125 mg/mL), respectively. However, ZnO showed a slightly lower MIC of 5.208 mg/mL against resistant co-culture than the WT co-culture with a MIC of 6.25 mg/mL (FIGS. 5A-5B).

TABLE 4
Minimum Inhibitory Concentration (MIC) values of Chitosan and ZnO against E. faecium
and E. coli.

| | Strains of MRFs and Wild Type Counterparts | | | | | |
|---|---|---|---|---|---|---|
| | | | | | | Co-culture |
| | | | | E. faecium | Co-culture | of MCC 13 |
| | | E. coli | | ATCC | of BAA | and ATCC |
| | E. coli | MCC 13 | E. faecium | 35667 | 2471 and | 35667 |
| Antimicrobials | BAA-2471 | (WT) | 1449 | (WT) | 1449 | (WT) |

MIC: mg/mL

| | | | | | | |
|---|---|---|---|---|---|---|
| Chitosan 1 | <0.0488 | Not | Not | Not | Not | Not |
| (3 kDA) | | detected (>50) | detected (>50) | detected (>50) | detected (>50) | detected (>50) |
| Chitosan 2 | 0.04883 | Not | Not | Not | Not | Not |
| (50 kDA) | | detected (>50) | detected (>50) | detected (>50) | detected (>50) | detected (>50) |
| ZnO | 13.54167 | 7.292 | 3.125 | 0.391 | 5.208 | 6.25 |
| C₁ZNPS | <0.0488 | 2.604 | 1.563 | 3.125 | 0.781 | 2.083 |
| C₂ZNPS | <0.0488 | 2.083 | 1.042 | 2.604 | 1.302 | 3.125 |

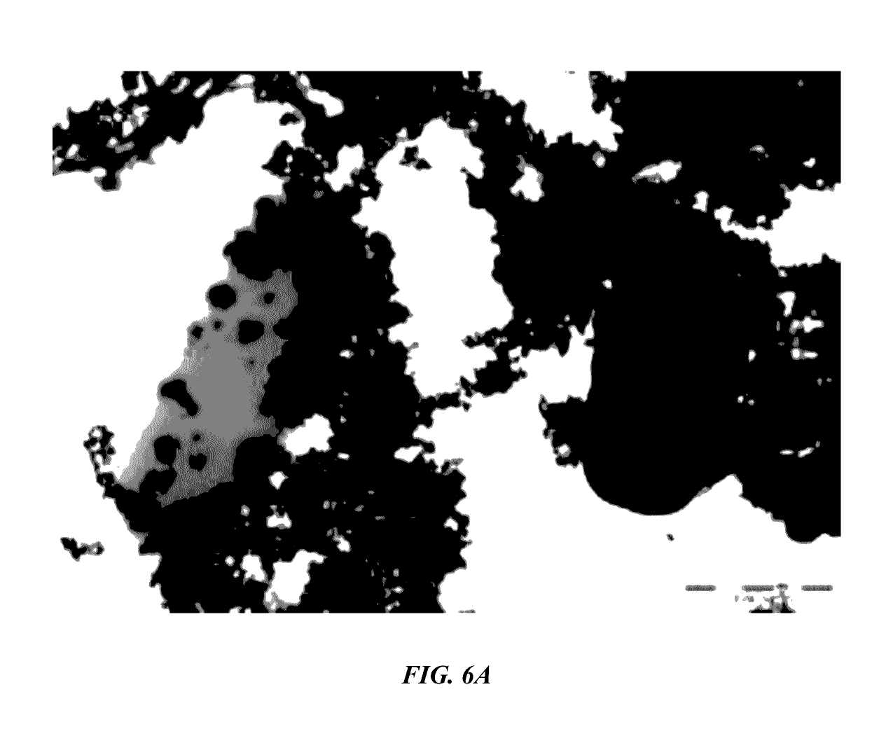

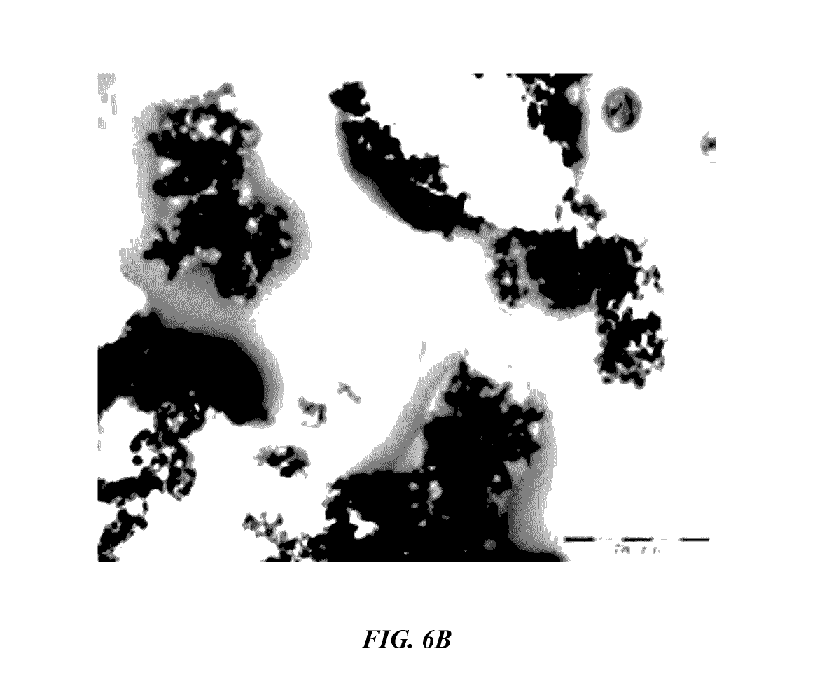

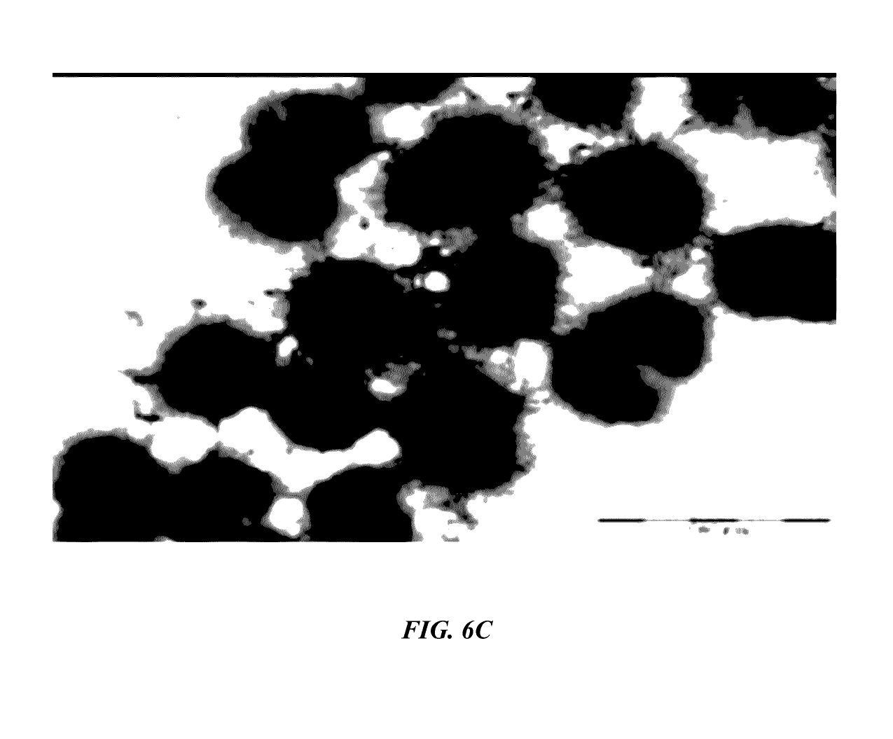

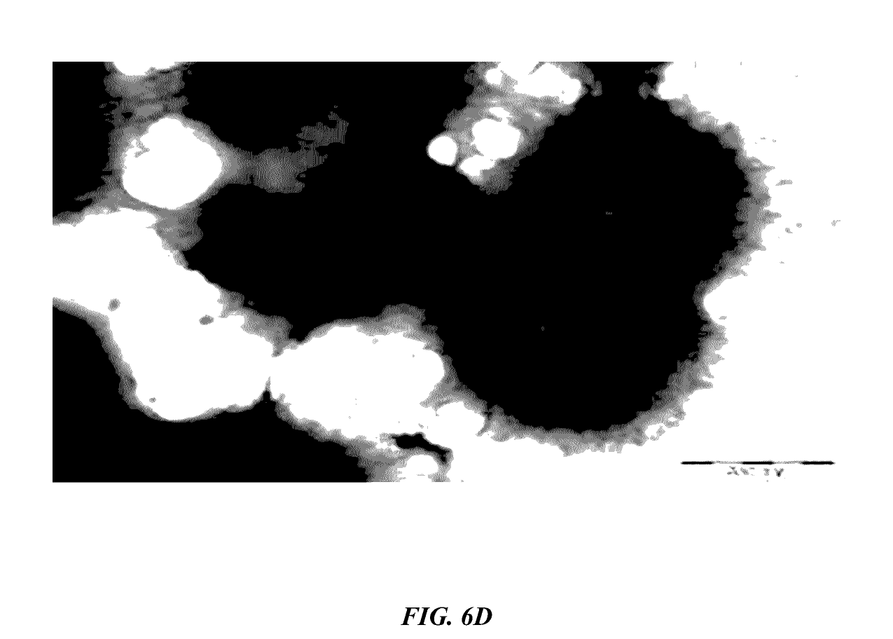

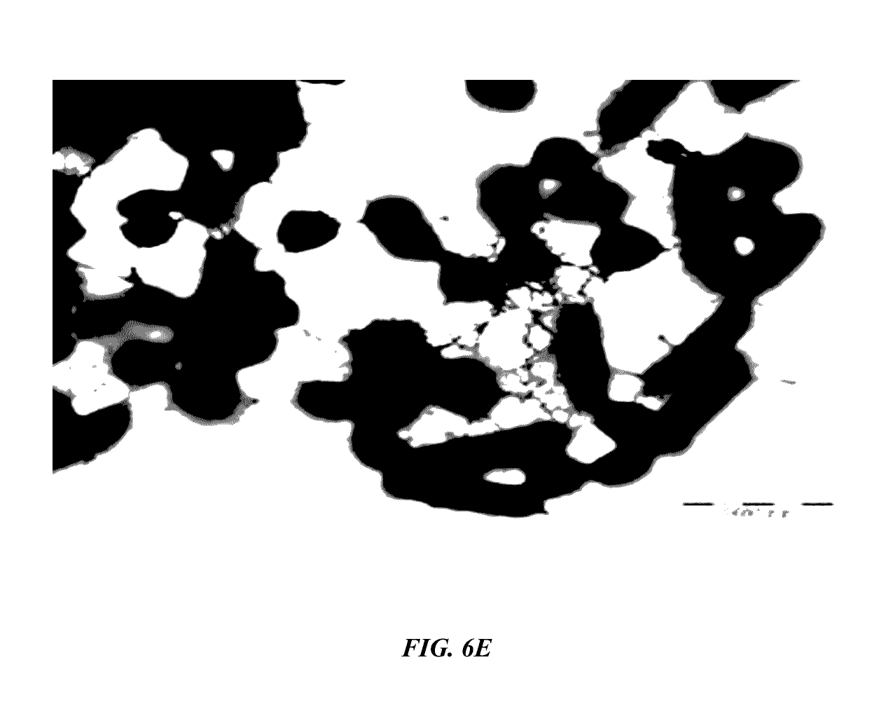

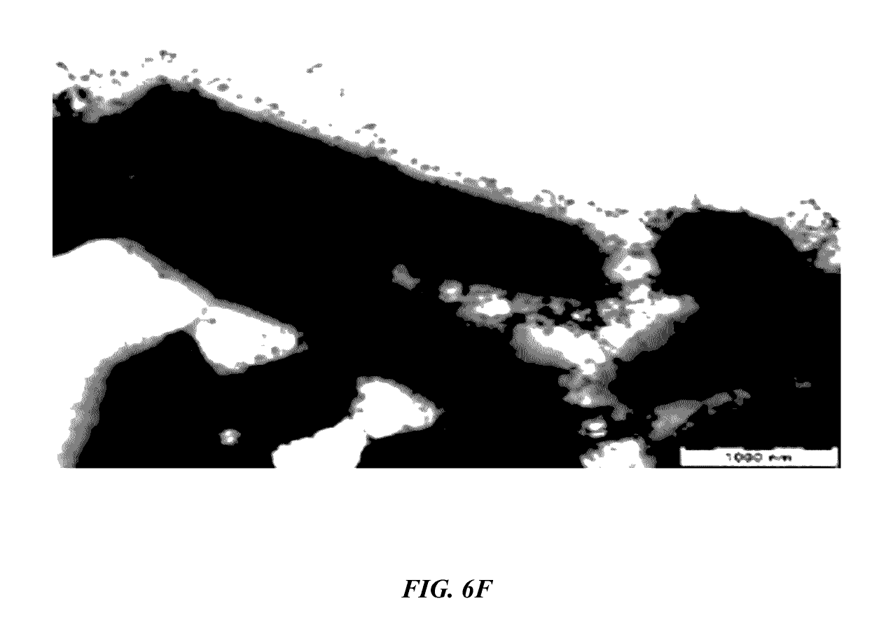

The antimicrobial efficacies of synergism of chitosan and ZnO tested after 24 and 48 hours incubation at 37° C. are shown in Table 4. Both C₁ZNPs and C₂ZNPs presented markedly higher antimicrobial efficacy against resistant co-cultures than WT co-cultures with a minimal average MIC of 0.781 compared to 2.083 mg/mL, respectively. Furthermore, the effectiveness of the CZNPs complex was closely related to the molecular weight of chitosan. Indeed, the MIC values of C₁ZNPs against resistant E. coli BAA-2471, E. faecium 1449 and co-culture were 0.0488, 1.563 and 0.781 mg/mL, respectively. However, the MIC values of C₂ZNPs against resistant E. coli BAA-2471, E. faecium 1449 and co-culture were 0.0488, 1.042 and 1.302 mg/mL, respectively. Interesting, C₁ZNPs with low MW (3 KDa) was much more effective than C₂ZNPs with high MW (50 KDa) against resistant co-culture. The MW was one of key factors of NPs to impact the antimicrobial efficacy against bacteria cultures (FIGS. 6A-6F).

The antimicrobial efficacies of chitosan tested after 24 hours incubation at 37° C. are shown in Table 4. The chitosan 1 and 2 was not effective against both E. faecium culture and the co-culture as well as their WT cultures. However, chitosan 1 and 2 showed a significantly inhibitory antimicrobial ability (0.0488 mg/mL) against multidrug resistant E. coli BAA-2471 culture compare to non-effective against WT E. coli MCC 13 culture.

The minimal inhibitory concentration (MIC) values of the antimicrobials tested after 24 hours incubation at 37° C. are shown in Table 4. Zinc oxide and chitosan were tested alone and in equal combined level (1:1) against separate cultures and co-cultures of E. coli strain ATCC BAA-2471 and E. faecium strain 1449. Chitosan 1, and 2, which vary in molecular weights (3 and 50 kDa, respectively) were ineffective at low concentrations (<5%) against either strains of bacteria as well as against the co-culture.

Zinc oxide only had relative effectiveness against E. faecium at a concentration of 3.125 mg/mL. Zinc oxide effectiveness at low concentrations against E. coli or against the co-culture was not detectible. Further tests can confirm if the co-cultures treated with zinc oxide inhibited only the growth of E. faecium or if both microbes grew relatively unhindered. The values denoted for combinatory antimicrobials are of total antimicrobial and therefore the concentration of each antimicrobial in these trials are half of the total concentration of antimicrobials. Detectable MICs were observed for the combination of antimicrobials against both separate cultures and the co-cultures. The C₂ZNPs was effective against E. coli and E. faecium alone with an MIC of 0.024 mg/mL and 12.5 mg/mL, respectively.

These trials give evidence for a synergistic property between the two antimicrobials as trials showed effective inhibition of E. coli, E. faecium, and the co-culture as compared to the trials using just one antimicrobial, which were not detected (>5%). The MIC of C₁ZNP against E. faecium was a fourth of the MIC of zinc oxide against E. faecium. The MIC of the combined antimicrobial C₁ZNPs and C₂ZNPs in the co-culture was twice that of the MIC needed against the separate cultures. These values give evidence that these MRF strains increase their resistance to combined chitosan and Zinc oxide when in the same culture. This gives evidence that the molecular weight of chitosan can greatly alter the effectiveness of the antimicrobial. Further tests can confirm the optimal molecular weight for this antimicrobial against a particular strain.

### D. DISCUSSION

This report assessed the potential for remediation of multi-drug resistant bacteria of two different nanoparticles, such as the chitosan and the ZnO. Although both of these have been evaluated previously either individually or in combination for their antibacterial properties, hitherto neither of these have been examined for potential for remediation of the multi-drug resistant bacteria.

Since the association between the molecular weight of chitosan and its antibacterial property has been controversial [32] and this has not been studied for MRF and VRE strains, investigations were initiated using two different chitosan nanoparticles, one oligomeric chitosan (3kDA) nanoparticles and a high molecular weight (50kDA) chitosan. Chitosan presents distinct mechanisms according to whether a bacterium is gram-positive or gram-negative; in previous studies, electron micrographs for S. aureus and E. coli interacting with chitosan show how the cell membrane of S. aureus was −weakened or even broken, while the cytoplasm of E. coli was concentrated and the interstice of the cell were clearly enlarged∥ [7], which provides ample evidence to suggest two main antibacterial mechanisms performed by chitosan. For gram-positive bacteria, chitosan forms a polymer membrane around the cell's surface preventing any nutrients from entering while for gram-negative bacteria, chitosan with lower molecular weight entered the cell through pervasion [6]. Chitosan seems to work more efficiently against gram-negative bacteria due to how its positively-charged structure adsorbs the electronegative substance in the cell and flocculate it to disturb the physiological activities of the bacteria and inhibit it.

However, chitosan is naturally a large molecule, therefore the use of chitosan oligomers presents a stronger choice if the molecules would be combined with another compound for a synergistic effect. Although chitosan oligomers do not have the same level of antibacterial activity as chitosan, their smaller size allows for a lower molecular weight and the facilitation of penetrating the bacterial surface. The effect of this property is seen as chitosan oligomers seemed to have an increased antibacterial activity against gram-negative bacteria at lower molecular weight such as 1 kDa, where Kyoon No et al., 2002 found that the growth of E. coli was reduced by 1 to 3 log cycles at a 1.0% concentration of chitosan oligomer with various degrees of polymerization [8]. Moreover, the ZnO nanoparticle effect is more pronounced “against gram-positive bacterial than gram-negative bacterial strains” [9]. Gram-positive bacteria have a thick layer of peptidoglycan polymer that encircles the cell and a much thicker cell wall, while gram-negative have two thin cell membranes divided into an outer membrane and a plasma membrane [2], therefore they both require different agents against their different structures. Chitosan oligomers' polysaccharide structure not only contain transcellular properties to cross cellular membranes, they also contain mucoadhesive and bioadhesive properties that contribute to their absorption improving effects [10]. The positively-charged chitosan does not only bind to negatively-charged oxides, it also can bind to different drugs or peptide hormones like calcitonin to deliver a specific dose effectively, partly due to its bioadhesive properties with gram-negative bacterial membranes or negative mucus in tissue membrane in the case of calcitonin delivery [10]. Since chitosan has the structural ability to bond to metal oxide compounds such as the ionic ZnO, a possibility exists to develop an antimicrobial agent that can efficiently work against both types of bacteria by bonding these two compounds in order to create a synergistic combination that targets a broader range of bacteria found in food products without risking human health. The applicability of ZnO as an antimicrobial agent is due to its morphology that broadens the uses of it against various bacteria. The compound's structure allows for an easier biocompatibility over other metal oxides, solubility in alkaline medium, and the Zn—O terminated polar surfaces [3]. Zinc oxide nanoparticles seem to inhibit or cause bacterial death more efficiently when they are smaller in size but higher in concentration. A smaller size at higher concentrations provides higher specific surface areas and facilitates the penetration of the antimicrobial agent into the bacterial membrane [5]. Virtually every unique property of ZnO proves to be beneficial in regards to antibacterial activity. Although ZnO is usually insoluble in water due to its high polarity, it can be managed efficiently in an aqueous cell culture media such as tryptic soy broth, also known as TSB. Zinc oxide possesses photo-oxidizing and photocatalysis impacts on chemical and biological species [2], which combined with its bio-safe composition, provides a safe interaction on food products where they come in contact with bacteria to inhibit and/or kill it to prevent food-related diseases.

Additionally, this study adds a novel perspective on the increased effectiveness of chitosan by molecular weight when in the presence of ZnO. While the oligomer of chitosan (3kDA) in literature shows the least potential as a possible effective antimicrobial, the effectiveness of chitosan oligomers against MRFs was substantially amplified by the addition of ZnO. The discrepancy between this data and the current literature may be due to the presence of ZnO along with the structural modifications of the chitosan oligomers used to generate optimal conditions.

Combination of chitosan oligomers with ZnO demonstrated synergistic effects in remediation of important food-borne bacteria including the resistant strains. Current literature has shown the synergistic antimicrobial properties of chitosan and ZnO [30], as well as detailed the synergistic properties of chitosan and ZnO individually against wild type bacteria [31]. However, there has been no quantitative data thus far on the precise concerted effect of these nanoparticles against multi-drug resistant microbes. This experiment has demonstrated the effect against both multi-drug resistant gram-positive and gram-negative fecal bacteria while comparing the combinatory antimicrobial properties to the individual nanoparticles. The overall objective of this experiment was to ascertain the efficacy of CZNPs synergism on co-cultured MRF strains through validated MIC tests and TEM assays. Specific goal was to validate a nanotherapeutic agent to further in situ intervention.

This research study has novelty demonstrated this success of CZNPs against both multi-drug resistant gram-positive and gram-negative fecal bacteria while comparing the combinatory antimicrobial properties to the individual nanoparticles. Synergism of CZNPs primarily C1ZNPs proved to be successfully suppressive to MRFs over WT strains. It is concluded that C1ZNPs has therapeutic potential to in situ intervention.

### E. REFERENCES

1. Bakhshi S, Padmanjali K S, Arya L S (2008) Infections in childhood acute lymphoblastic leukemia: an analysis of 222 febrile neutropenic episodes. Pediatr Hematol Oncol 25: 385-392.
2. Cattaneo C, Quaresmini G, Casari S, Capucci M A, Micheletti M, et al., (2008) Recent changes in bacterial epidemiology and the emergence of fluoroquinolone-resistant Escherichia coli among patients with haematological malignancies: results of a prospective study on 823 patients at a single institution. J Antimicrob Chemother 61: 721-728.
3. Kremery V, Spanik S, Mrazova M, Trupl J, Grausova S, et al., (2002) Bacteremias caused by Escherichia coli in cancer patients-analysis of 65 episodes. Int J Infect Dis 6:69-73.
4. Limayem A, Donofrio R S, Zhang C, Haller E, Johnson M G (2015) Studies on the drug resistance profile of Enterococcus faecium distributed from poultry retailers to hospitals, Journal of environmental science and health Part B Pesticides food contaminants and agricultural wastes. Accepted.
5. Centers for Disease Control and Prevention (CDC), (2013) Antibiotic Resistance Threats in the United States, 2013.
6. Lebreton F, van Schaik W, Manson McGuire A, Godfrey P, Griggs A, et al., (2013) Emergence of Epidemic Multidrug-Resistant Enterococcus faecium from Animal and Commensal Strains. mBio vol. 4 no. 4 e00534-13.
7. Eisenstein B I, Jones G W, (1988) The spectrum of infections and pathogenic mechanisms of Escherichia coli. Adv Intern Med 33: 231-252.
8. Johnson J R, Russo T A (2002) Extraintestinal pathogenic Escherichia coli: “the other bad E. coli”. J Lab Clin Med 139:155-162.
9. Orskov I, Orskov F (1985) Escherichia coli in extra-intestinal infections. J Hyg (Lond) 95: 551-575.
10. Johnson J R (1991) Virulence factors in Escherichia coli urinary tract infection. Clin Microbiol Rev 4: 80-128.
11. Johnson J R, Kuskowski M A, Gaj ewski A, Sahm D F, Karlowsky J A (2004) Virulence characteristics and phylogenetic background of multidrug-resistant and antimicrobial-susceptible clinical isolates of Escherichia coli from across the United States, 2000-2001. J Infect Dis 190: 1739-1744.
12. Johnson J R, Stell A L (2000) Extended virulence genotypes of Escherichia coli strains from patients with urosepsis in relation to phylogeny and host compromise. J Infect Dis 181: 261-272.
13. Franz C M, Huch M, Abriouel H, Holzapfel W, Galvez A (2011)
Enterococci as probiotics and their implications in food safety. Int J Food Microbiol 151: 125-140.
14. Werner G, Klare I, Heier H, Hinz K H, Bohme G, Wendt M, and Witte W (2000) Quinupristin/dalfopristin-resistant enterococci of the satA (vatD) and satG (vatE) genotypes from different ecological origins in Germany. Microb Drug Resist 6: 37-47.
15. Arias C A and Murray B E (2012) The rise of the Enterococcus: beyond vancomycin resistance. Nat Rev Microbiol 10: 266-278.
16. Limayem A (2015) The mutating gastrointestinal flora, multidrug resistant Enterococcus faecium, Agriculture, Food and Analytical Bacteriology. Accepted.
17. Laukova A, Chrastinova L, Placha I, Strompfova V, Szabóova R, et al., (2012) Beneficial effect of bacteriocin-producing strain Enterococcus faecium EF 55 of non rabbit origin in rabbits. In: Hacklander, K. and Thurner, C. Ed. BOKU-Reports on wildlife research and game management. Proc 4th World Lagomorph Conference, Vienna, Austria, Vienna, p. 77.
18. De Kwaadsteniet M, Todorov S D, Knoetze H, Dicks L M (2005), Characterization of a 3944 Da bacteriocin, produced by Enterococcus mundtii ST15, with activity against Gram-positive and Gram-negative bacteria. Int. J. Food Microbiol. 105:433-444.
19. Leroy F, Foulquie M R, Vuyst L D (2003) Enterococcus faecium RZS C5, an interesting bacteriocin producer to be used as a co-culture in food fermentation. Int. J. Food Microbiol. 88:235-240.
20. Lund B, Edlund C (2001) Probiotic Enterococcus faecium strain is a possible recipient of the vanA gene cluster. Clin. Infect. Dis. 32:1384-1385.
21. Fisher K, Phillips C (2009) The ecology, epidemiology and virulence of Enterococcus. Microbiol. 155:1749-1757.
22. Sahoo S K, Parveen S, Panda J J (2007) The present and future of nanotechnology in human health care. Nanomedicine 3:20-31.
23. Arias C A, Murray B E (2009) Antibiotic-resistant bugs in the 21st century—a clinical super-challenge. N Engl J Med 360:439-443.
24. Paul S, Seema N P (2015) In vitro antibacterial potential of chitosan and its derivatives on pathogenic enterobacteriaceae. Natl J Physiol Pharm Pharmacol 5:119-124.
25. Reddy K M, Feris K, Bell J, Wingett D G, Hanley C, Punnoose A (2007) Selective toxicity of zinc oxide nanoparticles to prokaryotic and eukaryotic systems. Appl Phys Lett 90:2139021-2139023.
26. Jin T, Sun D, Su J Y, Zhang H, Sue H J (2009) Antimicrobial efficacy of zinc oxide quantum dots against Listeria monocytogenes, Salmonella Enteritidis, and Escherichia coli O157:H7. J Food Sci 74:M46-52.
27. Dixit S et al., (2013) Phospholipid micelle encapsulated gadolinium oxide nanoparticles for imaging and gene delivery. RSC Adv 3:2727-2735.
28. Kumar A, Glaum M, El-Badri N, Mohapatra S, Haller E (2011) Initial observations of cell-mediated drug delivery to the deep lung. Cell Transplant 20:609-618.
29. Premanathan M, Karthikeyan K, Jeyasubramanian K, Manivannan G (2011) Selective toxicity of ZnO nanoparticles toward Gram-positive bacteria and cancer cells by apoptosis through lipid peroxidation. Nanomedicine. 7:184-92.
30. Elhady M M (2012) Preparation and characterization of chitosan/zinc oxide nanoparticles for imparting antimicrobial and U V protection to cotton fabric. International journal of carbohydrate chemistry, 6.
31. Perelshtein I, Ruderman E, Perkas N, Tzanov T, Beddow J, Joyce E, Gedanken, A (2013) Chitosan and chitosan-ZnO-based complex nanoparticles: formation, characterization, and antibacterial activity. J Mater Chem B 1:1968-1976.
32. No H K, Park N Y, Lee S H, Meyers S P (2002) Antibacterial activity of chitosans and chitosan oligomers with different molecular weights. Int J Food Microbiol 74:65-72.

All referenced publications are incorporated herein by reference in their entirety. Furthermore, where a definition or use of a term in a reference, which is incorporated by reference herein, is inconsistent or contrary to the definition of that term provided herein, the definition of that term provided herein applies and the definition of that term in the reference does not apply.

Experiment/Study 3 (Molecular Identification and Nanoremediation of Microbial Contaminants in Algal Systems Using Untreated Wastewater)

Wastewater-algal biomass is a promising option to biofuel production. However, microbial contaminants constitute a substantial barrier to algal biofuel yield. A series of algal strains, Nannochloris oculata and Chlorella vulgaris samples (n=30), were purchased from the University of Texas, and were used for both stock flask cultures and flat-panel vertical bioreactors. A number of media were used for isolation and differentiation of potential contaminants according to laboratory standards (CLSI). Conventional PCR amplification was performed followed by 16S rDNA sequencing to identify isolates at the species level. Nanotherapeutics involving a nanomicellar combination of natural chitosan and zinc oxide (CZNPs) were tested against the microbial lytic groups through Minimum Inhibitory Concentration (MIC) tests and Transmission Electronic Microscopy (TEM). Results indicated the presence of Pseudomonas spp., Bacillus pumilus/safensis, Cellulosimicrobium cellulans, Micrococcus luteus and Staphylococcus epidermidis strains at a substantial level in the wastewater-fed algal reactors. TEM confirmed the effectiveness of CZNPs on the lytic group while the average MICs (mg/mL) detected for the strains, Pseudomonas spp, Micrococcus luteus, and Bacillus pumilus were 0.417, 3.33, and 1.458, respectively. Conclusively, CZNP antimicrobials proved to be effective as inhibitory agents against currently identified lytic microbial group, did not impact algae cells, and shows promise for in situ interventions.

### A. INTRODUCTION

Rising concerns towards the global warming paradigm, along with potential scarcity from petroleum-derived gasoline and response by the U.S. to reduce gasoline consumption by 20% in 10 years, have renewed interest in sustainable biomass-based energy.^{[1,2]} Although lignocellulosic feedstock is anticipated as the key option to meet the Energy Independence and Security Act (EISA) plans of increasing renewable biofuels to at least 36 billion gallons by 2022 from 4.7 billion in 2007, there is an unmet need for additional cost-effective alternatives.^{[3,4]} Among the viable and scalable bio-resources for biofuel production, algae biomass is rising above other feedstock through its unique features. Unlike other natural feedstock, algae do not use fertile land and generate a higher yield per acre associated to an environmentally attractive CO₂ sequestration. It is also able to utilize brackish water with respect to the extensive range of by-products generated from bioethanol production.^{[5]} As such, algae biofuels have regained interest as a third generation bioethanol source that holds promise to accommodate the aims of Bioenergy and Technologies Offices (BETO). Interest in algae-based bioenergy has existed since 1978 in the U.S.^{[3,5]} However, optimizing the efficiency of algae biomass as a viable and scalable source enterprise remained limited during the last decade as the life cycle assessment (LCA) indicates algal biofuel production is not sustainable unless wastewaters are used for algae cultivation.^{[6]} Wastewaters provide delivery of nutrients such as phosphorus and nitrogen but are also hosts to microbial contaminants^{[7]} who are of prime concern in algae production that is performed under non-aseptic conditions. Bacteria including Proteobacteria, Bacteroidetes, and Actinobacteria would be detrimental to algal growth and can lead to a serious yield loss and subsequent erosion of profits from downstream by-products. Therefore, a novel natural antibiotic is of dire need for this valuable “green” industry.

Yield loss resulting from the lytic properties of bacterial contaminants have been reported as a major challenge which has been exacerbated due to requirement of an open non-sterile cultivation system for large-scale algae production.^{[8,9]} As such, the overall goal of the integrated system approach herein is to employ novel molecular and systemic methods to trace microbial contaminants from the source. This requires a complete screening of the predominant microbial groups in algal systems to ensure an efficient nanoremediation.

Chitosan is emerging as a strong natural antimicrobial, which is effective as an antibacterial agent against gram-negative strains and biofilms.^{[10]} Zinc oxide (ZnO) is also emerging as an effective nano-component for its potential to mitigate some gram-positive strains.^{[10]} The synergistic application of chitosan oligomers with zinc oxide nanoparticles show promise for a novel method that utilizes low molecular weight chitosan derivatives with the increased surface area of ZnO nanoparticles (CZNPs).^{[11]} The notable antimicrobial properties of ZnO are optimized by the use of chitosan oligomers that contain transcellular properties to cross cellular membranes^{[11,12]} its structural ability to bond to metal oxide compounds. Chitosan's mucoadhesive and bioadhesive properties with Gram-negative bacteria present a favorable opportunity to transport CZNPs to lytic microbial groups found in algal wastewater systems such as Pseudomonas spp., and several Bacillus strains.^{[12]} Together chitosan and ZnO work synergistically to target both Gram positive and Gram negative bacteria thus mimicking a broad spectrum antibiotic. Implementation of natural antimicrobial agents considered safe for human contact^{[11,13]} provide the necessary intervention for cost-effective, sustainable biofuel production. The objective of this study was to develop a comprehensive screening of the most prevalent microbial contaminants found in biofuel reactors and study the effectiveness of CZNPs against such contaminants. A complete identification of bacterial contaminants in algal biofuels would convey a greater insight for researchers to nanotherapeutic intervention.

### B. MATERIALS AND METHODS

i. Sample Collection

A series of algal strains namely, Nannochloris oculata Droop UTEX LB 1998 and Chlorella vulgaris Beijerink UTEX 259 samples were purchased from the Culture Collection of Algae at the University of Texas. The samples were used for both stock flask cultures and flat-panel vertical bioreactors in the biofuel lab at the Patel College of Global Sustainability at the University of South Florida, Tampa, Fla., USA.

ii. Differential/Selective Media Test

A number of selective and differential media such as tryptic soy agar (TSA), blood agar, starch agar, eosin-methylene blue (EMB) agar, MacConkey agar, salmonella-shigella (SS) agar, triple sugar iron (TSI) agar, xylose lysine deoxycholate (XLD) agar and mannitol salt agar (MSA) were used for isolation and differentiation of potential bacterial contaminants according to Clinical & Laboratory Standards Institute (CLSI) guidelines.

iii. PCR Assay and Gel Electrophoresis

DNA was extracted from isolated pure cultures using the QIAamp DNA Mini Kit (Qiagen, Valenica, Calif., USA) according to the manufacturer's instructions. The purity and yield of the extracted DNA was checked using the ND-1000 Nano-Drop spectrophotometer (Fisher Scientific, Pittsburgh, Pa., USA). Conventional PCR amplification targeting the 16S rRNA gene was performed using the following primer set: 8F: 5′-AGA GTT TGA TCC TGG CTC AG-3′ (SEQ ID NO:1), 1492R: 5′-GGT TAC CTT GTT ACG ACT T-3′ (SEQ ID NO:2). The PCR reaction was performed in a Mini-opticon with the following conditions: initial denaturation for 2 min at 94° C., 40 cycles of 1 min at 94° C. for denaturation, 1 min at 50° C. for annealing and 2 min at 72° C. for extension, a final extension of 4 min at 72° C., then 4° C. for holding the PCR products. 5 mL of amplified products along with 2 mL of 6× loading dye were stained with ethidium bromide and electrophoresed in a 2.0% agarose gel at 120 V for 15 min along with a 100 bp Ladder to verify product size. PCR products were further identified using 16S rRNA analysis (Table 5).

TABLE 5
Isolate identification at the genus-level by 16s rRNA sequencing.

| Tube ID | Pseudomonas |
|---|---|
| A | Pseudomonas |
| B | Pseudomonas |
| C | Pseudomonas |
| D | Pseudomonas |
| E | Pseudomonas |
| F | Pseudomonas |
| Q | Pseudomonas |
| R2 | Bacillus pumilus/Bacillus safensis |
| Pond 1 | Aeromonas |
| Pond 2 | Pseudomonas |
| Pond 3 | Pseudomonas |
| Pond 4 | Staphylococcus warneri/Staphylococcus |
| | pasteuri |
| Well 1 | Brevibacillus sp. |
| Well 2 | Bacillus sp. |
| H | Cellulosimicrobium cellulans |
| M | Micrococcus luteus |
| S | Staphylococcus epidermis |

iv. Preparation and Characterization of CZNPs

Medium molecular weight of chitosan powder (MW: 10 kDa; surface area: 4.56-0.74 m²/gL; surface porosity 89.3%; zeta potential: 36 mV-40 mV; acidity/basicity 6.2-7.0) along with hydrophobic zinc oxide (ZnO) powder (Surface area: 54 m²/g (MW: 20 nm); composition: 80.34% zinc and 19.6% oxygen; zeta potential: 9.5 mV-10 mV; acidity/basicity 6.5-7.0; crystallinity: Zincite, hexagonal wurtzite) were purchased from Sigma Aldrich, St. Louis, Mo. The degree of viscosity of chitosan were approximately 90% associated with hydrated and anhydrous crystallinity. The stock solution was prepared by dissolving spontaneously both agglomerate nanoparticle in 1% (v/v) of acetic acid 2.0 mg/mL and was adjusted pH using (NaOH) and stored at room temperature that should not exceed 25° C. The synthesis and purification of the CZNPs were performed based on Premanathan et al.^{[15]} and Zhang et al.^{[12]} protocols with some additional modifications. It includes the optimization of the core shell structure by designing a hydrophobic interior comprising ZnO with imaging moiety surrounded by an anionic functionalized lipid micellar (i.e., DOPA (1,2-dioleoyl-sn-glycero-3-phosphate); Avanti Polar lipid, Inc.) and chitosan coat shell.

v. Minimal Inhibitory Concentration (MIC) Assays

The lytic bacterial strains of Pseudomonas spp., Bacillus spp., and Micrococcus luteus cultured overnight were diluted 1 in 1,000 in fresh Tryptic Soy Broth (TSB) broth (Sigma, St. Louis, Mo., USA). A sterile 96-well microtiter plates were labeled. To all the categorized wells was added 50 mL of TSB. Each well was loaded with 50 mL of the bacterial suspension (5×108 CFU/mL) and screened against 1.0% CZNPs by 1:2 dilution to equal a total volume of 100 mL in each of the wells according to National Committee for Clinical Laboratory Standards definitions (NCCLS). All tests were performed in triplicate with identical results. The plates were incubated at 37° C. overnight and the MICS were determined after 24 h by visual determination of the minimum level of antimicrobial to inhibit growth. Inhibition of growth was determined by lack of turbidity in the wells.

vi. Microscopic Characterization Using Transmission Electron Microscopy (TEM)

Aliquots of bacteria in growth media were initially fixed in equal volume of 2% osmium tetroxide in distilled water for 1 h at 4° C. Following fixation, the bacteria were rinsed in distilled water and pelleted at 5000RPM for 10 min. This rinse step was repeated three times. A proper dilution of bacteria was obtained to yield approximately 2000-3000 bacteria per drop, and one drop of a sample was applied to a carbon-formvar coated copper grid. The grid was allowed to air dry. This procedure was repeated for each sample. Once dry, the grids were observed and photographed in an FEI Morgagni transmission electron microscope (FEI Corp., Hillsboro, Oreg., USA).

### C. RESULTS

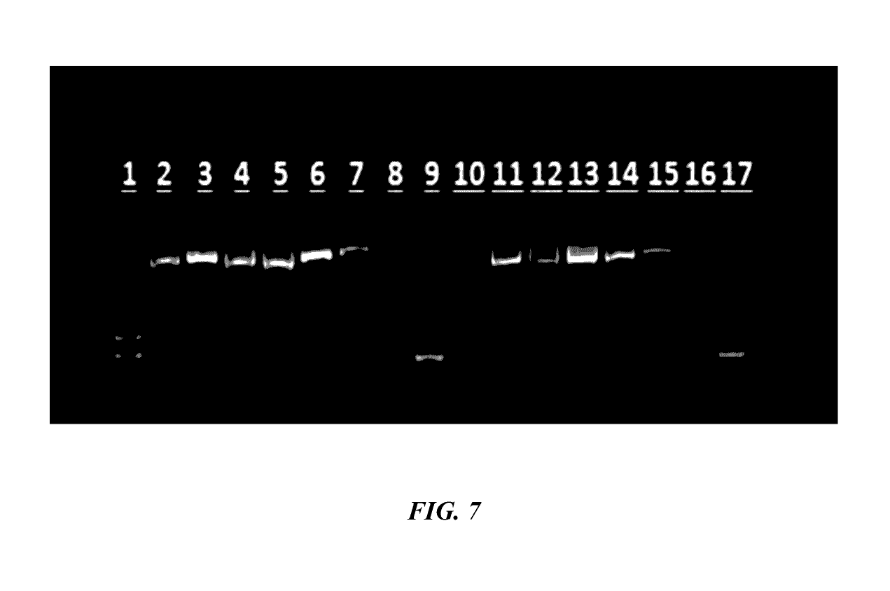

The preliminary findings have elucidated a wide range of microbial contaminants of approximately 20 microorganisms including fungi along with numerous rod shaped Gram-positive and Gram-negative bacteria. The presence of Pseudomonas as evidenced by colorless colonies on EMB and MacConkey agar was particularly interesting for its previously documented lytic effects on algal growth. The colorless and yellow colonies presented in MSA media suggested the appearance of Staphylococcus epidermidis and Micrococcus luteus, respectively.[14] Gel electrophoresis of amplified PCR products demonstrated distinct bands (FIG. 7) along with the absence of a band in the negative control indicates no degradation of target DNA and the absence of non-specific amplicons. The presence of strains presumptively identified using culture-based methods and some others that could not be identified was confirmed at the genus level by 16S rRNA sequencing as shown in Table 5. Many isolates were also identified at species level by further 16S rRNA sequencing as follows: Pseudomonas spp., Bacillus pumilus/safensis, Cellulosimicrobium cellulans, Micrococcus luteus and Staphylococcus epidermidis.

i. Microscopic Characterization Using TEM

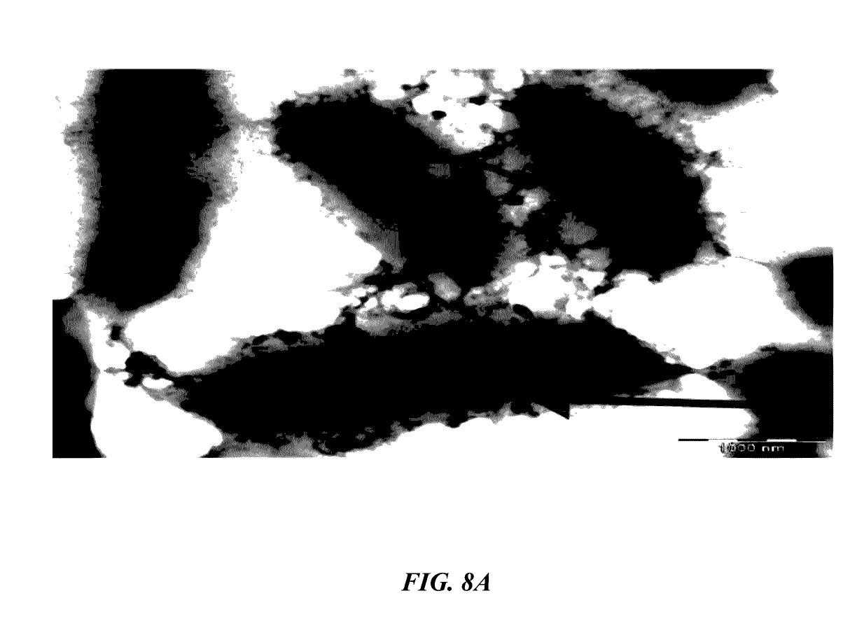

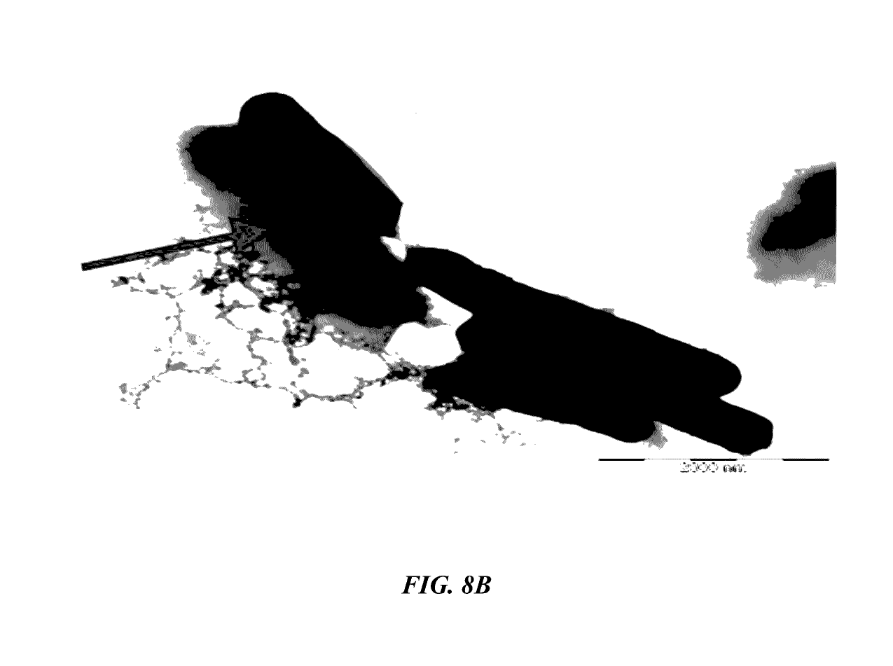

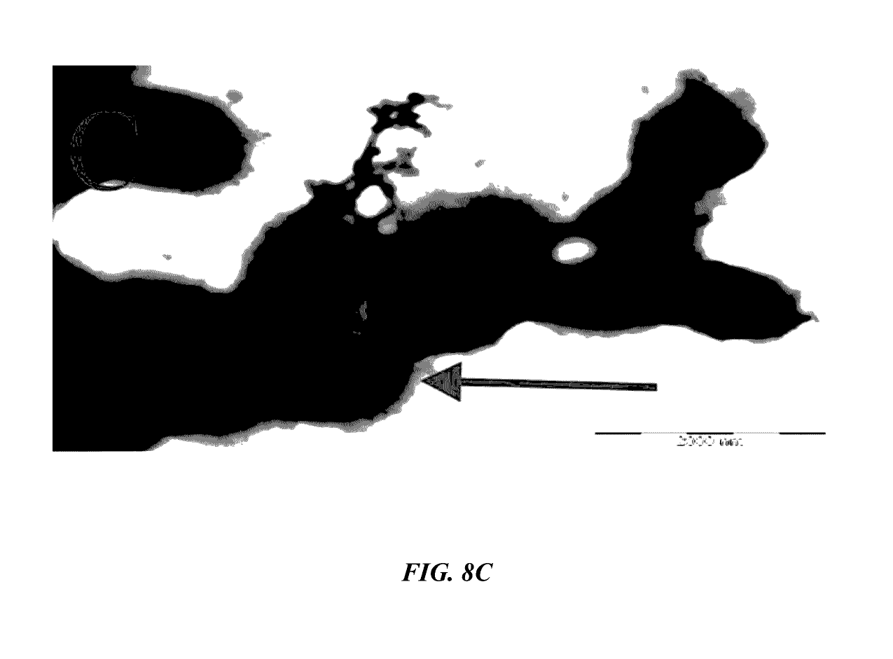

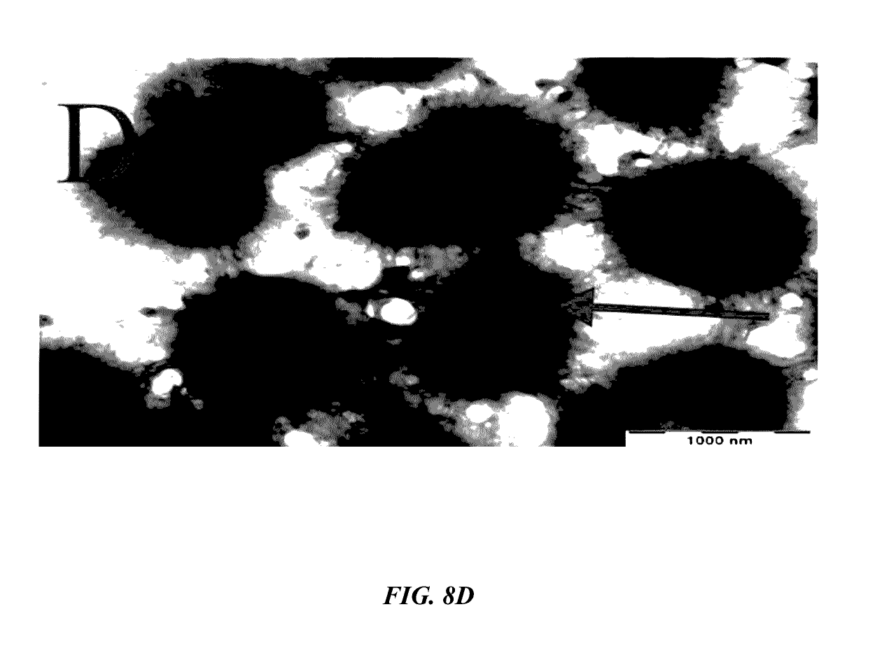

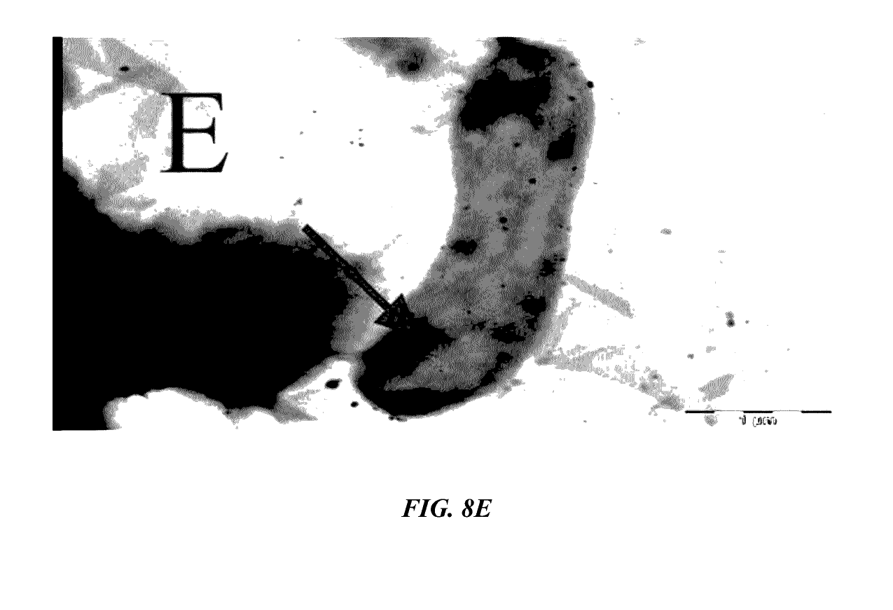

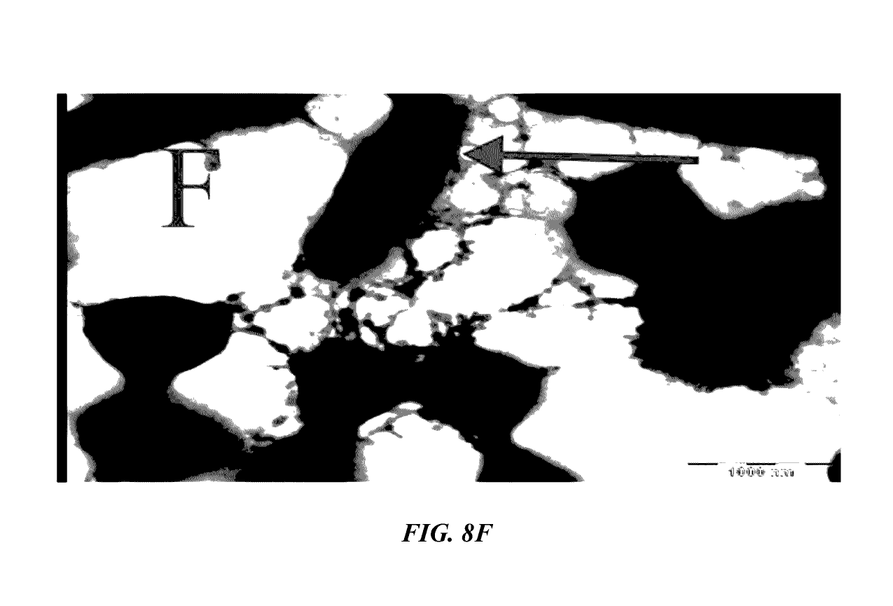

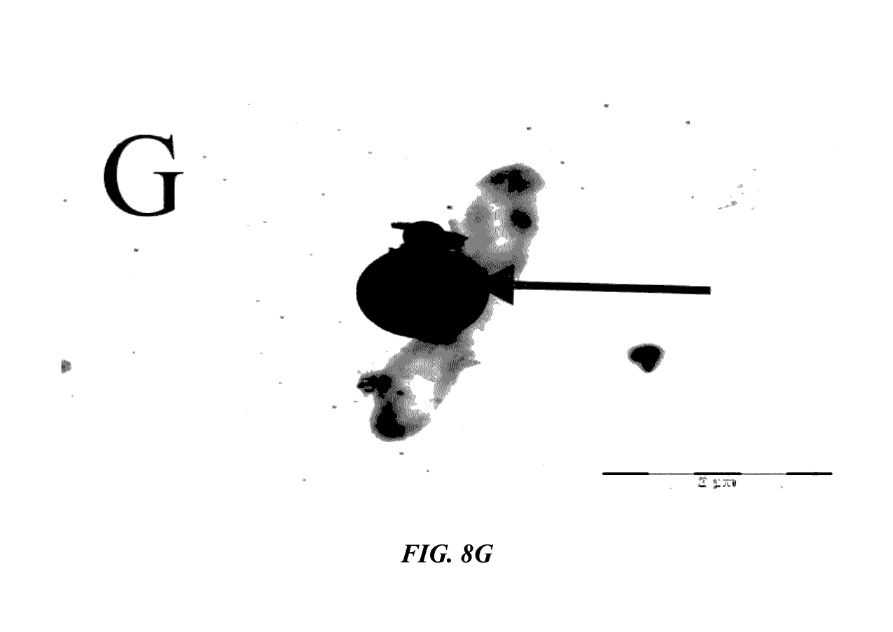

Identified strains of Pseudomonas, Micrococcus, and Bacillus were prepared for TEM to observe reaction against synergistic antimicrobial attack of chitosan (10 kDa) oligomers and ZnO nanoparticles after exposure for 24 h. The microscopy performed on the lytic bacteria revealed protrusions of microbial cell membranes with clear alterations caused by CZNPs in the bacteria's cell wall structure, cellular shape, and electron density compared to microbial control cells (FIGS. 8A-8G). Images for each bacterial group were collected to demonstrate the lytic effect CZNPs have on them, as can be seen in FIGS. 8A-8G.

ii. Minimum Inhibitory Concentration (MIC)

TEM confirmed the effectiveness of CZNPs on lytic groups Pseudomonas, Bacillus, and Micrococcus luteus (FIGS. 8A-8G) after 24 h of exposure. Cell death and lysis is observed through the contrast of low electron density in the bacterial cell as opposed to the high electron density of the invading nanoparticles. The antimicrobial effectiveness of chitosan and ZnO, separate and combined, after 24 h incubation at 37° C. are shown in Table 6. Every bacterial lytic group was exposed to an antimicrobial or combination of antimicrobials in triplicate and results shown are average between the trials. Antimicrobial susceptibility of the lytic groups against the individual application of chitosan 10 kDa provided similar inhibitory concentrations for Pseudomonas (2.083 mg/mL), Micrococcus luteus (2.083 mg/mL), and Bacillus (1.875 mg/mL). Zinc oxide was not effective against Pseudomonas, having an MIC test result of 8.333 mg/mL in contrast to its synergistic combination with chitosan that exhibited significant antimicrobial activity against the same bacteria with an MIC result of 0.417 mg/mL. Moreover, ZnO nanoparticles presented significant antimicrobial activity against Gram-positive Micrococcus (0.417 mg/mL) shown in Table 6. The MIC results demonstrated synergistic antimicrobial effect against all groups as the adhesive properties of chitosan with Gram-negative bacteria (Pseudomonas) enhance the delivery of nanoparticles across cellular membranes and has the ability to polymerize around the surface of Gram-positive bacteria to inhibit nutrient intake.^{[11]} Furthermore, ZnO nanoparticle effect are more pronounced against Gram-positive bacteria than Gram-negative bacterial strains,^{[15]} increasing the range of antimicrobial capacity of CZNPs. All MIC test results using chitosan-ZnO against Pseudomonas (0.417 mg/mL), Micrococcus luteus (3.333 mg/mL), and Bacillus (1.458 mg/mL) proved efficient when suspended in broth media (TSB).

TABLE 6
MIC values of (CZNPs) on lytic microbial contaminants.

| | MIC presented in mg/mL | | |
|---|---|---|---|
| Antimicrobials | Pseudomonas | Micrococcus luteus | Bacillus |
| Chitosan 1 (10 kDA) | 2.083 | 2.083 | 1.875 |
| Zinc Oxide | 8.333 | 0.417 | 1.250 |
| Zinc Oxide + | 0.417 | 3.333 | 1.458 |
| Chitosan 1 | | | |

### D. CONCLUSIONS

Several molecular studies have evidenced a broad continuum of microbial predators in algae fed wastewaters. Recent phylogenetic classifications elucidated the presence of primarily Bacteria and Archaea in anaerobically digested wastewaters designated for algae cultivation. Additional investigations revealed the existence of primarily Proteobacteria, Bacteroidetes, and Firmicutesphyla. However, there is a great need to determine an accurate identification of microbial contaminants at species level in each specific algae wastewater systems to reach an efficient large-scale nanoremediation. This research study has elucidated the presence of the lytic genera of Pseudomonas, Micrococcus, Staphylococcus and Bacillus detected at a substantial level in the wastewater-fed algal reactors for which the CZNPs complex revealed a promising nanoremedy that can be used in concentrations without affecting algae cells. Future research perspectives involving metagenomic analysis are envisioned. Reduction or elimination of contaminant bacteria is largely advantageous in terms of increase in algal yield with the added benefit of ensuring safe operation by protecting staff who work with such systems from contracting opportunistic bacterial infections. Therefore, an entire screening of the culturable and unculturable genetic materials directly recovered from the algae systems would be the option of choice to wastewater quality control optimization.

### E. REFERENCES

[1] Sivers, M.; Zacchi, G. Ethanol from lignocellulosics: A review of the economy. Bioresource Technol. 1996, 56, 131-140.
[2] Quinn, J.; Catton, K.; Wagner, N.; Bradley, T. Current large-scale us biofuel potential from microalgae cultivated in photobioreactors. BioEnergy Res. 2012, 5(1), 49-60.
[3] Reed, V. DOE biomass program. ABO Algae Biomass Summit 2012. [4] PEA; EISA E. Security Act of 2007. Public law. 2007, 110(140), 19.
[5] Aristidou, A.; Penttila, M. Metabolic engineering applications to renewable resource utilization. Curr. Opin. Biotechnol. 2000, 11, 187-198.
[6] ANL; NREL; PNNL. Renewable Diesel from Algal Lipids: Integrated Baseline for Cost, Emissions, and Resource Potential from a Harmonized Model. ANL/ESD/12-4; NREL/TP-5100-55431; PNNL-21437. 2012.
[7] Unnithan, V.; Unc, A.; Smith, G. B. Mini-review: A priori considerations for bacteria-algae interactions in algal biofuel systems receiving municipal wastewaters. Algal Res. 2014, 4, 35-40.
[8] Wang, H.; Zhang, W.; Chen, L.; Wang, J.; Liu, T. The contamination and control of biological pollutants in mass cultivation of microalgae. Bioresource Technol. 2013, 128, 745-750.
[9] Day, J.; Thomas, M.; Achilles-Day, U.; Leakey, R. Early detection of protozoan grazers in algal biofuel cultures. Bioresource Technol. 2012, 114, 715-719.
[10] Limayem, A.; Micciche, A.; Haller, E.; Zhang, C.; Mohapatra, S. Nanotherapeutics for mutating multi-drug resistant fecal bacteria. J. NanotecNanosci. 2015, 1, 100-106.
[11] Zheng, L.; Zhu, J. Study on antimicrobial activity of chitosan with different molecular weights. Carbohydrate Polym. 2003, 54(4), 527-530.
[12] Zhang, H.; Huang, X.; Sun, Y.; Xing, J.; Yamamoto, A.; Gao, Y. Absorption-improving effects of chitosan oligomers based on their mucoadhesive properties: A comparative study on the oral and pulmonary delivery of calcitonin. Drug Deliv. 2014, 21(6), 397-500.
[13] Sirelkhatim, A.; Mahmud, S.; Seeni, A.; Haida, N.; Chuo Ann, L.; Khadija, S.; Hasan, H.; Mohamad, D. Review on zinc oxide nanoparticles: Antibacterial activity and toxicity mechanism. Nano-Micro Lett. 2015, 7(3), 219-242.
[14] Gumbo, J. R.; Ross, G.; Cloete, T. E. The isolation and identification of predatory bacteria from a Microcystis algal Bloom. Afr. J. Biotechnol. 2010, 9, 663-671.
[15] Premanathan, M.; Karthikeyan, K.; Jeyasubramanian, K.; Manivannan, G. Selective toxicity of ZnO nanoparticles toward Gram-positive bacteria and cancer cells by apoptosis through lipid peroxidation. Nanomedicine 2011, 7, 184-192.
[16] Davis, R.; Fishman, D.; Frank, E.; Johnson, M.; Jones, S.; Kinchin, C.; Skaggsi, R.; Venteris, E.; Wigmostal, M. Integrated evaluation of cost, emissions, and resource potential for algal biofuels at the national scale. Environ. Sci. Technol. 2014, 48(10), 6035-6042.
[17] Wang, B. X.; Yang, X. R.; Lu, J. L.; Zhou, Y. Y.; Su, J. Q.; Tian, Y.; Zhang, J.; Wang, G. Z.; Zheng, T. L. 2010a. A marine bacterium producing protein with algicidal activity against Alexandrium tamarense. Harmful Algae 2010a, 13, 83-88.
[18] Wang, H.; Liu, L.; Liu, Z. P.; Qin, S. Investigations of the characteristics and mode of action of an algae lytic bacterium isolated from Tai Lake. J. Appl. Phycol. 2010b, 22, 473-478.
[19] Kong, M.; Chen, X; Xing, K.; Park, H. Antimicrobial properties of chitosan and mode of action: A state of the art review. Int. J. Food Microbiology 2010, 144(1), 51-63.
[20] Xie, Y.; He, Y.; Irwin, P.; Jin, T.; Shi, X. Antibacterial activity and mechanism of action of zinc oxide nanoparticles against Campylobacter jejuni. Appl. Environ. Microbiol. 2011, 77(7), 2325-2331.
[21] Premanathan, M.; Karthikeyan, K.; Jeyasubramanian, K.; Manivannan, G. Selective toxicity of ZNO nanoparticles toward Gram-positive bacteria and cancer cells by apoptosis through lipid peroxidation. Nanomedicine J. 2011, 7(2), 184-192.

All referenced publications are incorporated herein by reference in their entirety. Furthermore, where a definition or use of a term in a reference, which is incorporated by reference herein, is inconsistent or contrary to the definition of that term provided herein, the definition of that term provided herein applies and the definition of that term in the reference does not apply.

### DEFINITIONS

The term “administration” and variants thereof (e.g., “administering” a compound) in reference to a compound of the invention means introducing the compound or a prodrug of the compound into the system of the animal in need of treatment. When a compound of the invention or prodrug thereof is provided in combination with one or more other active agents (e.g., a cytotoxic agent, etc.), “administration” and its variants are each understood to include concurrent and sequential introduction of the compound or prodrug thereof and other agents.

As used herein, the term “composition” is intended to encompass a product comprising the specified ingredients, in the specified amounts, as well as any product which results, directly or indirectly, from combination of the specified ingredients in the specified amounts. Generally, the specified ingredients, or pharmaceutically acceptable salts and derivatives thereof, are suitable agents for use in the diagnosis, mitigation, treatment, cure, or prevention of disease in a subject, specifically but not exclusively effective in the treatment of nosocomial infections, when administered in an effective amount to a subject in need thereof.

As used herein, “patient”, “subject” and “subject in need of treatment” are used interchangeably to mean mammals in need of diagnosis or treatment of nosocomial infections.

The pharmaceutical compositions of the subject invention can be formulated according to known methods for preparing pharmaceutically useful compositions. Furthermore, as used herein, the phrase “pharmaceutically acceptable carrier” means any of the standard pharmaceutically acceptable carriers, such as a solvent, suspending agent or vehicle, for delivering the compound or compounds in question to the mammal. The carrier may be liquid or solid and is selected with the planned manner of administration in mind. The pharmaceutically acceptable carrier can include diluents, adjuvants, and vehicles, as well as implant carriers, and inert, non-toxic solid or liquid fillers, diluents, or encapsulating material that does not react with the active ingredients of the invention. Examples include, but are not limited to, phosphate buffered saline, liposomes, physiological saline, water, and emulsions, such as oil/water emulsions. The carrier can be a solvent or dispersing medium containing, for example, ethanol, polyol (for example, glycerol, propylene glycol, liquid polyethylene glycol, and the like), suitable mixtures thereof, and vegetable oils. The carrier can also include any and all other vehicles, coatings, diluents, antibacterial and antifungal agents, isotonic and absorption delaying agents, buffers, carrier solutions, suspensions, colloids, and the like. Formulations are described in a number of sources that are well known and readily available to those skilled in the art. Except insofar as any conventional media or agent is incompatible with the active ingredient, its use in the therapeutic compositions is contemplated. For example, Remington's Pharmaceutical Sciences (Martin E W [1995] Easton Pa., Mack Publishing Company, 19^{th} ed.) describes formulations which can be used in connection with the subject invention.

As used herein, the term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication commensurate with a reasonable benefit/risk ratio.

A “safe and effective amount” refers to the quantity of a component or composition that is sufficient to yield a desired therapeutic response without undue adverse side effects (such as toxicity, irritation, or allergic response) commensurate with a reasonable benefit/risk ratio when used in the manner of this invention for the treatment of nosocomial infections.

The term “therapeutically effective amount” as used herein means that amount of active compound or pharmaceutical agent that elicits the biological or medicinal response in a tissue, system, animal or human that is being sought by a researcher, veterinarian, medical doctor or other clinician. In reference to nosocomial infections or other gastrointestinal infections, an effective amount comprises an amount sufficient to cause the infection to subside or heal and/or to decrease the growth rate of the infection. In some embodiments, an effective amount is an amount sufficient to delay development. In some embodiments, an effective amount is an amount sufficient to prevent or delay occurrence and/or recurrence. An effective amount can be administered in one or more doses.

As used herein, “treatment” refers to obtaining beneficial or desired clinical results, or any measurable mitigation of disease in a subject, including resolution, reduction, halting progression, and/or slowing progression of a disease. Beneficial or desired clinical results include, but are not limited to, any one or more of the following: alleviation of one or more symptoms, diminishment of extent of infection, stabilized (i.e., not worsening) state of infection, preventing or delaying spread (e.g., metastasis) of the infection, preventing or delaying occurrence or recurrence of the infection, delay or slowing of infection progression, amelioration of the infection state. The methods of the invention contemplate any one or more of these aspects of treatment.

The advantages set forth above, and those made apparent from the foregoing description, are efficiently attained. Since certain changes may be made in the above construction without departing from the scope of the invention, it is intended that all matters contained in the foregoing description or shown in the accompanying drawings shall be interpreted as illustrative and not in a limiting sense.

It is also to be understood that the following claims are intended to cover all of the generic and specific features of the invention herein described, and all statements of the scope of the invention that, as a matter of language, might be said to fall therebetween.

What is claimed is:

1. A nanoparticle formulation for treating a nosocomial infection, comprising a combination of a therapeutically effective amount of chitosan and a therapeutically effective amount of zinc oxide.

2. A methodology for treating a nosocomial infection, comprising administering a combination of a therapeutically effective amount of chitosan and a therapeutically effective amount of zinc oxide.
